SEQUENCE LISTING (17 sequences)

SEQ ID NO: 1 - protein, 7 aa, synthetic construct
GFTFSSY

SEQ ID NO: 2 - protein, 6 aa, synthetic construct
SSGGTY

SEQ ID NO: 3 - protein, 10 aa, synthetic construct
GGTTVVGFDY

SEQ ID NO: 4 - protein, 15 aa, synthetic construct
RASESVDNYGISFMN

SEQ ID NO: 5 - protein, 7 aa, synthetic construct
AASNQGS

SEQ ID NO: 6 - protein, 9 aa, synthetic construct
QQSKEVPYT

SEQ ID NO: 7 - protein, 217 aa, synthetic construct
DIVLTQSPASLAVSLGQRATISCRASESVDNYGISFMNWFQQKPGQPPKLLIYAASNQGGVPARFSGSGSGTDFSLNIHPMEEDDTAMYFCQQSKEVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 443 aa, synthetic construct
DVKLVESGGGLVKPGGSLKLSCAASGFTFSSYIMSWVRQTPEKRLEWVATISSGGTYTYYPDSVKGRFTISRDNAKNTLYLQMSSLKSEDTAMYYCTRGGTTVVGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - protein, 660 aa, synthetic construct
DIVLTQSPASLAVSLGQRATISCRASESVDNYGISFMNWFQQKPGQPPKLLIYAASNQGGVPARFSGSGSGTDFSLNIHPMEEDDTAMYFCQQSKEVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECDVKLVESGGGLVKPGGSLKLSCAASGFTFSSYIMSWVRQTPEKRLEWVATISSGGTYTYYPDSVKGRFTISRDNAKNTLYLQMSSLKSEDTAMYYCTRGGTTVVGFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 10 - protein, 20 aa, Homo sapiens
GLGLPLIDEVVSPEPEPLNT

SEQ ID NO: 11 - protein, 310 aa, Homo sapiens
MGLCKCPKRKVTNLFCFEHRVNVCEHCLVANHAKCIVQSYLQWLQDSDYNPNCRLCNIPLASRETTRLVCYDLFHWACLNERAAQLPRNTAPAGYQCPSCNGPIFPPTNLAGPVASALREKLATVNWARAGLGLPLIDEVVSPEPEPLNTSDFSDWSSFNASSTPGPEEVDSASAAPAFYSQAPRPPASPGRPEQHTVIHMGNPEPLTHAPRKVYDTRDDDRTPGLHGDCDDDKYRRRPALGWLARLLRSRAGSRKRPLTLLQRAGLLLLLGLLGFLALLALMSRLGRAAADSDPNLDPLMNPHIRVGPS

SEQ ID NO: 12 - protein, 16 aa, Homo sapiens
LLLGLLGFLALLALMS

SEQ ID NO: 13 - protein, 16 aa, Homo sapiens
SRETTRLVCYDLFHWA

SEQ ID NO: 14 - protein, 16 aa, Homo sapiens
EPLTHAPRKVYDTRDD

SEQ ID NO: 15 - protein, 16 aa, Homo sapiens
RTPGLHGDCDDDKYRR

SEQ ID NO: 16 - protein, 16 aa, Homo sapiens
SDPNLDPLMNPHIRVG

SEQ ID NO: 17 - protein, 16 aa, Homo sapiens
ALGWLARLLRSRAGSR

(19) United States
(12) Patent Application Publication
(10) Pub. No.: US 2026/0337257 A1
(43) Pub. Date: Oct. 29, 2026

(54) Method of Diagnosing and Treating Prostate Cancer Using Zinc Finger Protein-Like 1

(71) Applicant: University of Louisiana Monroe, Monroe, LA (US)

(72) Inventor: Girish V. Shah, W. Monroe, LA (US)

(73) Assignee: University of Louisiana Monroe, Monroe, LA (US)

(21) Appl. No.: 19/104,405

(22) PCT Filed: Aug. 18, 2023
(86) PCT No.: PCT/US2023/072500

§ 371 Date: Feb. 18, 2025

Related U.S. Application Data
(60) Provisional application No. 63/371,902, filed on Aug. 19, 2022.
(60) Provisional application No. 63/387,115, filed on Dec. 13, 2022.

(51) Int. Cl. C07K 16/18; A61K 39/00; A61P 35/00; G01N 33/575

(52) U.S. Cl. CPC C07K 16/18; A61P 35/00; G01N 33/5758; A61K 2039/505

(57) ABSTRACT

Disclosed herein is a method of diagnosis and treatment of prostate cancer using an antibody for zinc finger protein-like 1 (ZFPL1). The method may comprise obtaining a serum sample from the patient, detecting whether zinc finger protein-like 1 (ZFPL1) is present in the serum sample, diagnosing the patient with prostate cancer when the presence of ZFPL1 in the serum sample is detected, and then administering an effective amount of anti-ZFPL1 antibodies to the diagnosed patient.

### CROSS-REFERENCE TO RELATED APPLICATIONS

This application is a 371 of PCT Application Serial No. PCT/US23/72500, filed Aug. 18, 2023, which claims the benefit of priority from U.S. Provisional Patent Application Ser. No. 63/371,902, filed on Aug. 19, 2022, and entitled “Neuroendocrine Marker (NEM) for Prostate Cancer Diagnosis and Monitoring and the Method of Measuring the Same.” This application also claims the benefit of priority from U.S. Provisional Patent Application Ser. No. 63/387,115, filed on Dec. 13, 2022, and entitled “Neuroendocrine Marker (NEM) for Prostate Cancer Diagnosis and Monitoring and the Method of Measuring the Same,” all of which are hereby incorporated by reference in their entirety.

### REFERENCE TO A SEQUENCE LISTING XML

This application contains a Sequence Listing which has been submitted electronically in XML format. The Sequence Listing XML is incorporated herein by reference. Said XML file, created on Aug. 2, 2023, is named 019976-187142-00_SL.xml and is 17,392 bytes in size.

### BACKGROUND

Prostate cancer is the second most common cancer and the sixth leading cause of cancer death among men worldwide. Prostate cancer displays tremendous diversity in its characteristics from a slow-growing tumor of little clinical significance to an aggressively metastatic disease. This provides an enormous opportunity to identify multiple biomarkers representing different stages of cancer progression. Unfortunately, prostate-specific antigen (PSA) is the only established blood biomarker for multiple purposes including prostate cancer detection, stratification of patients into prognostic risk groups, determination of overall tumor burden, and tracking of response to a local or systemic treatment. Moreover, the prognosis of this disease is still assessed with routine pathological parameters such as Gleason score, number or percentage of positive cores and the maximum percentage of tumor involvement in any core.

PSA is a kallikrein protease produced predominantly by luminal cells of the prostate, but also secreted in small amounts by the pancreas and the uterus. PSA is not cancer-specific but is produced normally in the prostate; its levels increase in prostate cancer as well as several benign conditions such as benign prostatic hyperplasia (BPH) and prostate inflammation. As a result, the serum PSA test nonspecifically detects many benign conditions as well as many prostate tumors that are low-grade and thus indolent. Therefore, PSA-based diagnosis requires confirmation by invasive, repetitive and costly procedures such as transrectal ultrasound-guided biopsy. On the other hand, ~15% of prostate cancer cases display low or normal serum PSA levels, a majority of which are highly aggressive with neuroendocrine (NE) features suggesting that the PSA test may not detect all lethal prostate cancers requiring aggressive treatment.

It has been reported that calcitonin (CT) and its receptors (CTR) are expressed selectively by basal but not secretory cells of benign prostate epithelium. However, all cells of malignant prostate epithelium express CT and CTR, and their expression increases with tumor progression. Moreover, the activation of CT-CTR axis induces an invasive phenotype in benign prostate cells. To date, a non-invasive method for accurately diagnosing prostate cancer in a patient has remained elusive.

### SUMMARY

The invention disclosed herein is directed to method of diagnosing and treating prostate cancer by targeting zinc finger protein-like 1 (ZFPL1). Through the process of subtraction hybridization, ZFPL1 has been identified as being selectively expressed only in malignant—but not in benign—prostate cells. It has been further discovered that the ZFPL1 protein plays a role in tumor development. Thus, treatments that target ZFPL1 are effective at treating prostate cancer.

An immunosensor for detecting zinc finger protein-like 1 (ZFPL1) in a biological sample embodying features of the present invention may comprise an anti-ZFPL1 monoclonal antibody immobilized on a substrate. The monoclonal antibody specifically binds to a ZFPL1 protein that comprises an epitope selected from a group consisting of SEQ ID No: 10, SEQ ID No: 12, SEQ ID No: 13, SEQ ID No: 14, SEQ ID No: 15, SEQ ID No: 16, or SEQ ID No: 17.

In another embodiment of the immunosensor, the monoclonal antibody comprises a heavy chain variable region (HCVR) comprising heavy chain complementarity-determining regions (CDRs), the heavy chain CDRs comprising SEQ ID Nos: 1, 2, and 3; and a light chain variable region (LCVR) comprising light chain CDRs, the light chain CDRs comprising SEQ ID Nos: 4, 5, and 6. In another embodiment, the monoclonal antibody comprises the HCVR (SEQ ID NO:7) and the LCVR (SEQ ID NO:8). In another embodiment, the isotype of the monoclonal antibody is immunoglobulin G (IgG). In another embodiment, the antibody has an equilibrium dissociation constant (KD) value of 100 nM and a half maximal inhibitory concentration (IC50) of 10 nM.

In another embodiment of the immunosensor, the light source generates white-light and the optical sensor is configured to detect light (inclusively) within the 500 nm to 900 nm wavelength range. In another embodiment, the immunosensor further includes a processor that calculates a frequency shift between a first sensor reading of the substrate with substantially no ZFPL1 and a second sensor reading with ZFPL1 bound to the monoclonal antibodies. In another embodiment, the first sensor reading is where less than 5% of the monoclonal antibodies are bound to ZFPL1 and the second sensor reading is where more than 5% of the monoclonal antibodies are bound to ZFPL1. In another embodiment, the frequency shift is detected at a ZFPL1 concentration in the biological sample of less than 1 μg/ml.

An immunoassay method for detecting prostate cancer in a biological sample in accordance with the present invention may comprise a first step of contacting the biological sample with a monoclonal antibody immobilized on a substrate and a second step of detecting the presence of ZFPL1 in the biological sample. The monoclonal antibody specifically binds to a ZFPL1 protein that comprises an epitope selected from a group consisting of SEQ ID No: 10, SEQ ID No: 12, SEQ ID No: 13, SEQ ID No: 14, SEQ ID No: 15, SEQ ID No: 16, or SEQ ID No: 17.

In another embodiment of the immunoassay method, the monoclonal antibody comprises an HCVR comprising heavy chain CDRs, the heavy chain CDRs comprising SEQ ID Nos: 1, 2, and 3; and an LCVR comprising light chain CDRs, the light chain CDRs comprising SEQ ID Nos: 4, 5, and 6. In another embodiment, the monoclonal antibody comprises the HCVR (SEQ ID No:7) and the LCVR (SEQ ID NO:8). In another embodiment, the isotype of the monoclonal antibody is immunoglobulin G (IgG). In another embodiment, the antibody has an equilibrium dissociation constant (KD) value of 100 nM and a half maximal inhibitory concentration (IC50) of 10 nM.

In another embodiment of the immunoassay method, the presence of the ZFPL1 in the biological sample is detected at a ZFPL1 concentration of less than 1 μg/ml. In another embodiment, the step of contacting the biological sample with a monoclonal antibody immobilized on a substrate comprises an incubation period of at least 30, 45, 60, 90, 120 or 180 minutes. In another embodiment, the step of detecting the presence of the ZFPL1 in the biological sample comprises conducting at least one of a chemiluminescent assay, an immunofluorescent assay, enzyme-linked immunosorbent assay (ELISA), radioimmunoassay, a Western blot assay, an enzyme immunoassay, an immunoprecipitation assay, an immunohistochemical assay, an immunochromatographic assay, a dot blot assay, a slot blot assay, a lateral flow assay, an optical immunoassay. In another embodiment, the step of detecting the presence of the ZFPL1 in the biological sample comprises conducting a label-free optical immunoassay. In another embodiment, the method further comprises obtaining a first reading before contacting the biological sample with the monoclonal antibody immobilized on the substrate and obtaining a second reading after contacting the biological sample with the monoclonal antibody immobilized on the substrate, wherein the step of obtaining a reading comprises: collimating a white-light source by a lens to illuminate the monoclonal antibody immobilized on the substrate and using an optical detector to detect transducing signals, comprising reflected optical interference fringes. In another embodiment, the step of detecting the presence of the ZFPL1 in the biological sample comprises comparing the first reading to the second reading and measuring a shift in frequency. In another embodiment, data peaks ranging from 550 nm to 750 nm are used to measure the frequency shift.

A method of diagnosing prostate cancer embodying features of the present invention may comprise the steps of first, obtaining a serum sample from the patient; second, detecting whether ZFPL1 is present in the serum sample; and third, diagnosing the patient with prostate cancer when the presence of ZFPL1 in the serum sample is detected. More specifically, this method can be accomplished by contacting the serum sample with a monoclonal antibody specific to ZFPL1 that is immobilized on a substrate, detecting the specific binding between the monoclonal antibody and the ZFPL1, determining the level of ZFPL1 in the patient sample, and identifying the patient as having a malignant prostate tumor when the level of ZFPL1 is above a baseline level of ZFPL1 observed in corresponding healthy subjects. The monoclonal antibody specifically binds to a ZFPL1 protein that comprises an epitope selected from a group consisting of SEQ ID No: 10, SEQ ID No: 12, SEQ ID No: 13, SEQ ID No: 14, SEQ ID No: 15, SEQ ID No: 16, or SEQ ID No: 17.

In another embodiment of the method of diagnosis, the monoclonal antibody comprises an HCVR comprising heavy chain CDRs, the heavy chain CDRs comprising SEQ ID Nos: 1, 2, and 3; and an LCVR comprising light chain CDRs, the light chain CDRs comprising SEQ ID Nos: 4, 5, and 6. In another embodiment, the monoclonal antibody comprises the HCVR (SEQ ID No:7) and the LCVR (SEQ ID NO:8). In another embodiment, the isotype of the monoclonal antibody is immunoglobulin G (IgG). In another embodiment, the antibody has an equilibrium dissociation constant (KD) value of 100 nM and a half maximal inhibitory concentration (IC50) of 10 nM. In another embodiment, the level of the ZFPL1 is determined using a label-free optical immunoassay. In another embodiment, the baseline level of ZFPL1 in the serum sample is 3.3 ng/mL.

A method of diagnosing and treating prostate cancer embodying features of the present invention may comprise the steps of first, obtaining a serum sample from the patient; second, detecting whether zinc finger protein-like 1 (ZFPL1) is present in the serum sample, third, diagnosing the patient with prostate cancer when the presence of ZFPL1 in the serum sample is detected; and fourth, administering an effective amount of anti-ZFPL1 antibodies to the diagnosed patient. The monoclonal antibody specifically binds to a ZFPL1 protein that comprises an epitope selected from a group consisting of SEQ ID No: 10, SEQ ID No: 12, SEQ ID No: 13, SEQ ID No: 14, SEQ ID No: 15, SEQ ID No: 16, or SEQ ID No: 17.

In another embodiment of the method of diagnosis, the monoclonal antibody comprises an HCVR comprising heavy chain CDRs, the heavy chain CDRs comprising SEQ ID Nos: 1, 2, and 3; and an LCVR comprising light chain CDRs, the light chain CDRs comprising SEQ ID Nos: 4, 5, and 6. In another embodiment, the monoclonal antibody comprises the HCVR (SEQ ID No:7) and the LCVR (SEQ ID NO:8). In another embodiment, the isotype of the monoclonal antibody is immunoglobulin G (IgG). In another embodiment, the antibody has an equilibrium dissociation constant (KD) value of 100 nM and a half maximal inhibitory concentration (IC50) of 10 nM. In another embodiment, the level of the ZFPL1 is determined using a label-free optical immunoassay. In another embodiment, the baseline level of ZFPL1 in the serum sample is 3.3 ng/mL.

In another embodiment, the monoclonal antibody treatment is administered intravenously, subcutaneously, or intraperitoneally. In another embodiment, the monoclonal antibody treatment leads to at least one effect selected from the group consisting of inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, and stable disease.

A method of treating cancer in a human subject embodying features of the present invention may comprise the administration of an effective amount of anti-ZFPL1 antibodies to a patient suffering from prostate cancer. The antibody specifically binds to a protein that comprises an epitope selected from a group consisting of SEQ ID No: 10, SEQ ID No: 12, SEQ ID No: 13, SEQ ID No: 14, SEQ ID No: 15, SEQ ID No: 16, or SEQ ID No: 17.

In another embodiment of the method of treatment, the anti-ZFPL1 antibody is a monoclonal antibody, an antigen-binding fragment thereof, or a protein ligand. In another embodiment, the anti-ZFPL1 antibody binds to at least four nucleotides within the nucleotide positions of 62-77, 127-284 or 293-308 within SEQ ID NO: 11. In another embodiment, the anti-ZFPL1 antibody binds to at least seven nucleotides within the nucleotide positions of 62-77, 127-284 or 293-308 within SEQ ID NO: 11. In another embodiment, the anti-ZFPL1 antibody is a chimeric or humanized antibody. In another embodiment, the anti-ZFPL1 antibody comprises a variant Fc domain.

In another embodiment, the monoclonal antibody treatment is administered intravenously, subcutaneously, or intraperitoneally. In another embodiment, the monoclonal antibody treatment leads to at least one effect selected from the group consisting of inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, and stable disease.

In another embodiment of the method of treatment, the effective amount of the anti-ZFPL1 antibody is between 0.5 and 5.0 mg/kg body weight of the patient. In another embodiment, the treatment is administered for 2 or 3 consecutive days and then the administration is ceased for at least three weeks. In another embodiment, after three weeks, the human's serum is tested for the presence of the protein of sequence SEQ ID NO: 11. In another embodiment, the treatment is administered for a second 2 or 3 consecutive days if the protein of sequence SEQ ID NO: 11 is present in concentrations above 3 ng/ml.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the following figures, ZFPL1 refers to zinc finger protein like 1; PC refers to prostate cancer; CT refers to calcitonin; BPH refers to benign prostatic hyperplasia; IHC refers to immunohistochemistry; ICC refers to immunocytochemistry; RT-qPCR refers to reverse transcription-quantitative PCR; OV refers to overexpression; DEX refers to dexamethasone; si-refers to small interfering; and p-refers to phosphorylated.

FIG. 1A is representative photomicrographs that show the presence of amplified ZFPL1 mRNA in PC3M, DU145, LNCaP, M1 (stably expressing inactive CTR), C4, PC3-CTR, PC3 prostate cancer cells following qRT-PCR reaction. FIG. 1B is a bar graph that shows quantitative representation of the ZFPL1 gene expression bands normalized with GAPDH housekeeping gene. FIG. 1C is a representative photomicrograph depicting the ZFPL1 product size band of 34.1 kDa on an immunoblot. FIG. 1D is a bar graph representing the mean relative ZFPL1 mRNA abundance±SEM (n=3) in LNCaP-C4 cells after treatment with CT at 0, 5, 10, 50 and 100 nM concentrations. FIG. 1E is a bar graph representing the mean relative ZFPL1 mRNA abundance±SEM (n=3) in PC3-CTR cells after treatment with CT at 0, 5, 10, 50 and 100 nM concentrations. FIG. 1F is a bar graph representing dose-dependent increase in relative ZFPL1 mRNA abundance in LNCaP-C4 cells (mean±SEM of n=3) in response to synthetic androgen R1881.

FIG. 2A is a bar graph that presents the mean±SEM (n=6) percentage of ZFPL1 immunopositive cell populations per field (magnification, ×400) in various normal human organs. FIG. 2B is representative photomicrographs of ZFPL1-immunopositive cells in normal human organ sections showing ZFPL1-immunopositive cells along with a normal prostate, which is ZFPL1 immuno-negative. FIG. 2C is a bar graph that presents the relative ZFPL1 mRNA abundance in normal, BPH, and prostate cancer specimens with different Gleason scores. FIG. 2D illustrates data extracted from TCGA and Oncomine portals showing upregulation of ZFPL1 gene expression in prostate cancer specimens.

FIG. 3A depicts photomicrographs demonstrating the specificity of in situ hybridization after a prostate cancer specimen was treated with sense ZFPL1 siRNA probe (left) or antisense ZFPL1 siRNA probe (right). FIG. 3B depicts photomicrographs of ZFPL1 mRNA expression in prostate sections of different cancer stages in comparison with non-cancer specimens. The left panel of FIG. 3C is representative photomicrographs that show the presence of ZFPL1-immunopositive cells (red) in prostate cancer tissue (left) vs matched normal tissue (right) by immunofluorescence. The nuclear stain is DAPI (blue). The right panel of FIG. 3C is a bar graph that represents the mean percentage (n=6) of ZFPL1 immunopositive cells per field (magnification, ×400) in the prostate cancer tissue vs matched normal tissue test. In FIG. 3D, the representative photomicrographs on the left show H&E staining of human prostate cancer tissue sample (dark bluish staining is for nuclear hematoxylin and pink staining is for eosin) while the photomicrographs on the right show green immunofluorescent labeling for ZFPL1 and blue color labeling for nuclear DAPI. White arrows depict cancerous areas for corresponding staining. FIG. 3E is representative photomicrographs that reveal ZFPL1-immunopsitive cells (red) and nuclear DAPI (blue) in different samples of a prostate cancer tissue microarray. The bar graph in FIG. 3F presents the quantitated data of the prostate cancer tissue microarray of FIG. 3E.

In FIG. 4A, the representative photomicrographs show colocalization of ZFPL1 and chromogranin A (CgA) in PC3-CTR cells (upper panels) and human prostate cancer tissue (lower panels) evaluated by immunofluorescence technique. In FIG. 4B, the representative photomicrographs show colocalization of ZFPL1 and CD44 in PC3-CTR cells (upper panels) and human prostate cancer tissue (lower panels) evaluated by immunofluorescence technique.

FIG. 5A is representative photomicrographs that show the colocalization of ZFPL1 (green) and exosome CD81 (red) in PC3-CTR and LNCaP PC cells. FIG. 5B is representative photomicrograph that reveal the colocalization of ZFPL1 (green) and exosome/secretosome maker CD63 (red). FIG. 5C is representative photomicrographs that show the colocalization of ZFPL1 (green) and Golgi body marker GM130 (red). FIG. 5D is a representative immunoblot that illustrates the co-precipitation of CD81 with ZFPL1 in the exosomal isolates of PC3-CTR and LNCaP-C4 PC cells.

FIG. 6A shows immunoblots that demonstrate the comparative efficacy of three siRNAs against ZFPL1 to suppress ZFPL1 protein levels in PC3-CTR and LNCaP-C4 cells by western blot analysis. In FIG. 6B, an immunoblot demonstrated that the transfection of ZFPL1 expression plasmid in PC3-CTR and LNCaP-C4 cells led to an increase in ZFPL1 protein levels in both cell lines.

FIG. 7A is a bar graph showing the effect of +10 nM CT on proliferation of PC-3CTR cells that received either non-sense siRNA or ZFPL1 siRNA. The representative photomicrographs in FIG. 7B demonstrate the effect of either non-sense (control) or ZFPL1 siRNA (1, 2 or 3)±CT on cleaved caspase 3 expression in PC3-CTR (upper panel) and LNCaP-C4 (lower panel) cells. The bar graphs of FIG. 7C present the pooled data of four separate experiments performed with LNCaP-C4 and PC3-CTR cell lines. In FIG. 7D, the first four pairs of photomicrographs show the expression of cleaved caspase 3 (green) in PC3-CTR and LNCaP-C4 cells expressing carrier plasmid. The next four pairs of photomicrographs reveal the expression of cleaved caspase 3 in PC3-CTR and LNCaP-C4 cells overexpressing ZFPL1.

FIG. 7E shows bar graphs that present the pooled data of the four separate experiments of FIG. D. In FIG. 7F, representative photomicrographs show the localization of cleaved caspase-3 staining in the nuclei of LNCaP-C4 cells.

The representative photomicrographs in FIG. 8A show the effect of ±10 nM CT on invasiveness of PC3-CTR cells receiving either non-sense siRNA or ZFPL1 siRNA (1, 2 or 3) FIG. 8B shows two bar graphs that summarize the pooled data of the experiments of FIG. 8A. The representative photomicrographs in FIG. 8C show the effect of ±10 nM CT on invasiveness of LNCaP-C4 and PC3-CTR cells expressing either carrier pCMV5-XL4 plasmid or the plasmid with ZFPL1 expression plasmid. FIG. 8D shows two bar graphs that summarize the pooled data of the experiments of FIG. 8C. Next, FIG. 8E shows representative photomicrographs of wound healing assays for cell migration of PC3-CTR cells transfected with ZDPL1 siRNA3 (siRNA-Row 2) or ZFPL1 expression vector (OVER-Row 4) and treated with ±CT (10 nM). FIG. 8F shows a bar graph that summarizes the pooled data of the experiments of FIG. 8E.

The representative immunoblots of FIG. 9A show the effect of ±10 nM CT on p-Akt473 and p-Akt308 proteins in PC3-CTR cells receiving either non-sense (control) siRNA or ZFPL1 siRNA1, ZFPL siRNA2, or ZFPL1 siRNA3. Also included in FIG. 9A are the normalized bar graphs (pAkt/total Akt) of densitometric quantitation of the immunoblots. The representative immunoblots of FIG. 9B show the effect of ±10 nM CT on p-Akt473 and p-Akt308 proteins in LNCaP-C4 cells receiving either non-sense (control) siRNA or ZFPL1 siRNA1, ZFPL siRNA2 or ZFPL1 siRNA3. Also included in FIG. 9B are the normalized bar graphs (p-Akt/total Akt) of densitometric quantitation of the immunoblots.

The representative immunoblots of FIG. 9C show the effect of ±10 nM CT on p-Akt473 and p-Akt308 proteins in PC3-CTR cells transfected with either carrier plasmid or ZFPL1 expression plasmid, respectively. Also included in FIG. 9C are the normalized bar graphs (pAkt/total Akt) of densitometric quantitation of the immunoblots. The representative immunoblots of FIG. 9D show the effect of ±10 nM CT on p-Akt473 and p-Akt308 proteins in LNCaP-C4 cells transfected with either carrier plasmid or ZFPL1 expression plasmid, respectively. Also included in FIG. 9D are the normalized bar graphs (p-Akt/total Akt) of densitometric quantitation of the immunoblots.

The representative photomicrographs in FIG. 9E show the effect of ±10 nM CT on pAkt staining in LNCaP-C4 and PC3-CTR cells receiving either non-sense or ZFPL1 siRNA (1, 2 or 3). Scale bar=50 μm. FIG. 9F is two bar graphs summarizing the pooled data of four separate experiments of with PC3-CTR and LNCaP-C4 cells receiving non-sense or ZFPL1 siRNAs. The representative photomicrographs of FIG. 9G show the effect of ±10 nM CT on p-Akt-immunopositive cells per field (magnification, ×400; green) in PC3-CTR cells expressing either carrier plasmid or ZFPL-overexpression plasmid. Scale bar=50 μm. FIG. 9H is two bar graphs summarizing the pooled data of four separate experiments of with PC3-CTR and LNCaP-C4 cells expressing either carrier plasmid (C) or ZFPL1 overexpression plasmid (OV). In FIG. 9I, representative photomicrographs at higher magnification (×1,000) show the nuclear localization of pAKT (green).

FIG. 10 is a scattergram presenting the serum profiles of ZFPL1 and PSA in healthy donors and positively confirmed patients with prostate cancer.

FIG. 11 is one embodiment of a ZFPL1 immunosensor.

FIG. 12A is a graph that demonstrates the ZFPL1 wavelength shifts recorded by the immunosensor of FIG. 11, and FIG. 12B is a graph that demonstrates the BSA wavelength shifts recorded by the immunosensor of FIG. 11.

FIG. 13A is a ZFPL1 calibration curve for an ELISA test, and FIG. 13B is a ZFPL1 calibration curve for the immunosensor of FIG. 11.

In FIG. 14A, the graph on the left is a Receiver Operating Characteristic Curve of ZFPL1's negative and positive predictability for prostate cancer, and the graph on the right is a corresponding prediction curve for normal (0) vs prostate cancer (1). In FIG. 14B, the graph on the left is a Receiver Operating Characteristic Curve of PSA's negative and positive predictability for prostate cancer, and the graph on the right is a corresponding prediction curve for normal (0) vs prostate cancer (1). In FIG. 14C, the graph on the left is a Receiver Operating Characteristic Curve of ZFPL1+PSA's negative and positive predictability for prostate cancer, and the graph on the right is a corresponding prediction curve for normal (0) vs prostate cancer (1).

In FIG. 15A, the graph on the left is a Receiver Operating Characteristic Curve of ZFPL1's negative and positive predictability for prostate cancer in the gray zone, and the graph on the right is a corresponding prediction curve for normal (0) vs prostate cancer (1). In FIG. 15B, the graph on the left is a Receiver Operating Characteristic Curve of PSA's negative and positive predictability for prostate cancer in the gray zone, and the graph on the right is a corresponding prediction curve for normal (0) vs prostate cancer (1).

FIG. 16 is a line graph representing the effect of ZFPL1 monoclonal antibody on PC3-CTR and DU145 cell lines.

FIG. 17 is a scatterplot of tumor volume over time in animal model treatment experiments.

FIG. 18 is representative images of tumors (untreated and treated) and a graph of their weights at the time of necropsy.

### DETAILED DESCRIPTION

Detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. Alternate embodiments may be devised without departing from the spirit or the scope of the invention. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward.

As used herein, the terms “a” or “an” are defined as one or more than one. The term “plurality,” as used herein, is defined as two or more than two. The term “another,” as used herein, is defined as at least a second or more. The terms “comprises,” “comprising,” or any other variation thereof are intended to cover a non-exclusive inclusion, such that a process, method, article, or apparatus that comprises a list of elements does not include only those elements, but may include, other elements not expressly listed or inherent to such process, method, article, or apparatus. An element proceeded by “comprises” does not, without more constraints, preclude the existence of additional identical elements in the process, method, article, or apparatus that comprises the element. The terms “including,” “having,” or “featuring,” as used herein, are defined as comprising (i.e., open language). The term “coupled,” as used herein, is defined as connected, although not necessarily directly, and not necessarily mechanically. As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. Relational terms such as first and second, top and bottom, right and left, and the like may be used solely to distinguish one entity or action from another entity or action without necessarily requiring or implying any actual such relationship or order between such entities or actions.

Discovery of prostate cancer marker: Prostate cancer is the most common visceral cancer diagnosed in men. A successful management of a prostate cancer patient largely depends on cancer detection before the cancer has metastasized. Although the serum PSA screening has improved the detection of this disease in an early stage, it has been observed that this test is not reliable, and a positive result must be confirmed with costly, repetitive and invasive TRUS-guided biopsy. This is because PSA is a natural product of a normal prostate gland and is found in sera of healthy individuals as well as cancer patients. Therefore, the inclusion of a new marker found only in the prostates of cancer patients should enhance the specificity and the precision of prostate cancer detection, reducing the need for diagnostic biopsies.

The inventors have discovered that Zinc finger protein-like 1 (ZFPL1) is a novel prostate tumor-specific protein that co-localizes with chromogranin A (a marker for neuroendocrine differentiation) and CD44 (a marker of cancer stem cells) in prostate cancer cells, suggesting that ZFPL1 provides a measure of neuroendocrine population of a prostate tumor. Since neuroendocrine as well as stern cell phenotypes are associated with castration-resistant metastatic cancer cells, the new marker should not only detect cancer at an early stage, but can also provide its future course, especially its potential with respect to the capacity to grow rapidly and metastasize. In short, the data presented herein suggests that this new marker will help identify the prostate cancer patients with aggressive phenotypes.

This new evidence demonstrates that ZFPL1-immunopostive cell populations are selectively localized to the malignant part of the prostate. ZFPL1-positive cells increase with increase in tumor grade and Gleason scores. ZFPL1 is secreted in blood through exosomes, and serum ZFPL1 levels in cancer patients are several-folds higher than those in age-matched normal individuals. Analysis of serum samples from over 100 patients suggest that ZFPL1 was more reliable than PSA in true prostate cancer detection, and could differentiate cancer patients from those with no cancer in the gray zone (patients displaying serum PSA levels in the range of 4-10 ng/ml). These results demonstrate that the ZFPL1 test remarkably increases the specificity, efficacy, and precision of prostate cancer detection as compared to PSA alone.

Based on these discoveries, a ZFPL1-specific antibody was created against synthetic peptide GLGLPLIDEV VSPEPEPLNT (SEQ ID NO 10), one of the epitopes of ZFPL1. This antibody was then used to develop a novel immunosensor-based assay for prostate cancer diagnosis. Finally, both in vitro and in vivo experimentation was conducted to study the efficacy of the inventive ZFPL1 antibody as a treatment for prostate cancer. The results of the study show that the antibody significantly slowed tumor growth.

Discovery/Characterization of Neuroendocrine Marker: Studies have reported a remarkable up-regulation of calcitonin (CT) and/or its receptors in malignant prostates. Moreover, the activation of CT-CTR axis induces an invasive phenotype in benign prostate cells. In contrast, the knock-down of CT/CTR induces the loss of invasive phenotype in aggressive prostate cancer cells. To identify the key factors associated with CT-CTR axis-induced increase in tumorigenicity and metastasizing capacity of prostate cancer cells, the inventors identified nine CT-responsive genes from a prostate cancer cDNA library by subtraction hybridization (Table 1). Among those, the inventors further characterized one protein, ZFPL1, which was most prevalent in prostate cancer cDNA library among the nine CT-responsive genes:

TABLE 1

| Name of identified CT-responsive genes | | Signal Strength |
|---|---|---|
| Immunoglobulin Heavy Constant Gamma 1 | IGHG1 | 16994+ |
| Calpain 2 | CAPN2 | 15935++ |
| S100 Calcium Binding Protein A 11 | S100A11 | 14304.5+ |
| F-Box Protein 2 | FBXO2 | 14211.5+++ |
| Zinc Finger Protein Like 1 | ZFPL1 | 12559.5++++ |
| ZFP36 Ring Finger Protein Like 1 | ZFP36L1 | 11487+++ |
| Immunoglobulin Heavy Variable 4-31 | IGHV431 | 10901+ |
| Branched Chain Ketoacid Dehydrogenase | BCKDK | 10102+ |
| Kinase | | |
| Butyrophilin Subfamily 2 Member A2 | BTN2A2 | 13006++ |

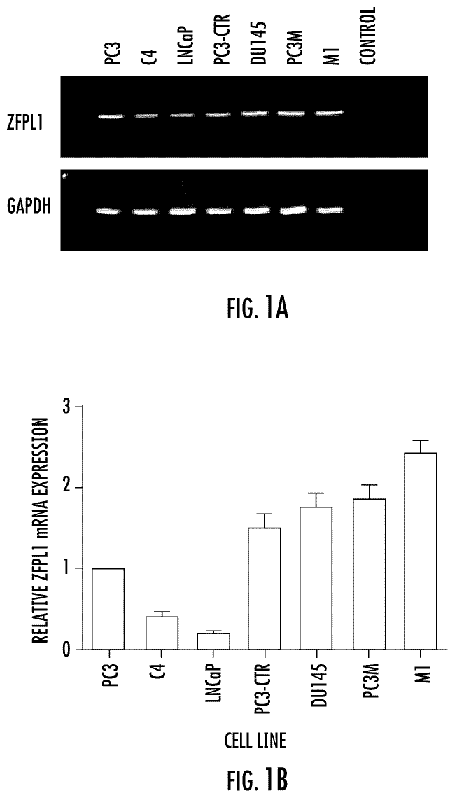

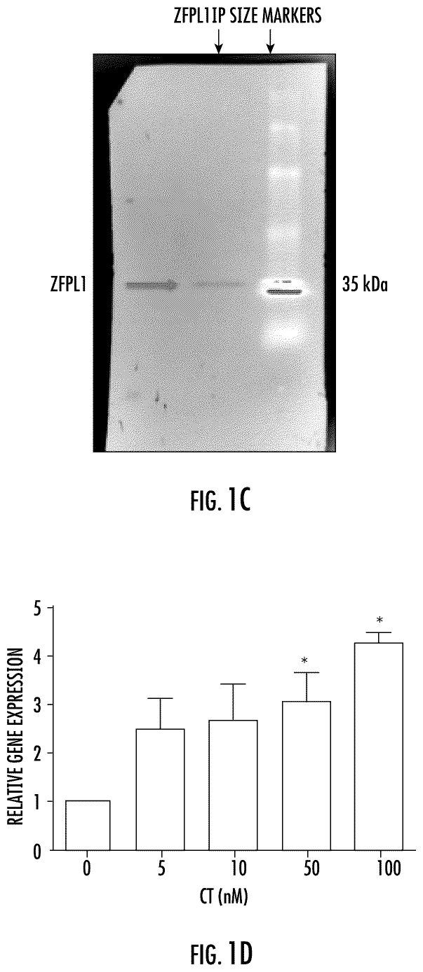

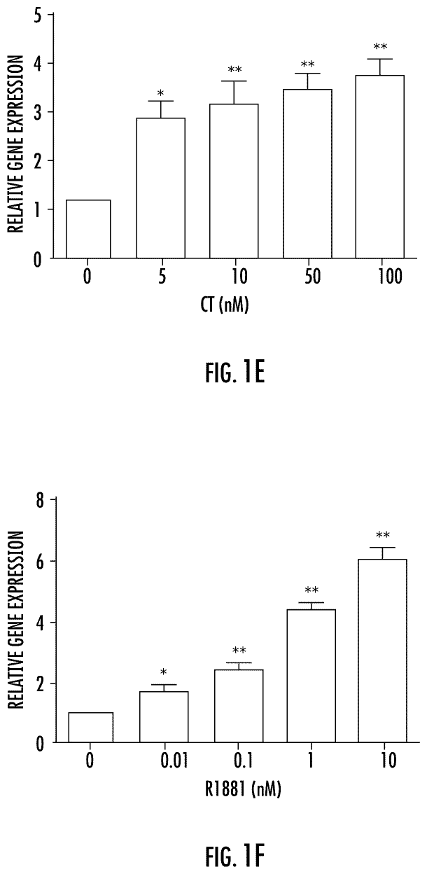

Expression of ZFPL1 mRNA in prostate cancer cell lines: In FIG. 1A, the relative abundance of ZFPL1 mRNA in multiple prostate cancer cell lines (LNCaP, PC3, PC-3M and LNCaP-C4) was determined by RT-qPCR. FIG. 1B displays the results normalized by GAPDH mRNA levels. The abundance of ZFPL1 mRNA in PC cell lines was compared with that of PC3 (which was set at 1). Among the cell lines studied, PC3-CTR, DU145 and PC3M cell lines displayed comparable ZFPL1 mRNA levels, but they were higher than those of PC3 cells. By contrast, the ZFPL1 mRNA abundance was remarkably lower in LNCaP and LNCaP-C4 cells compared with that of PC3 cells. Notably, M1 cell line (which expressed negative mutant CT receptor) demonstrated the highest ZFPL1 mRNA abundance.

Turning now to FIG. 1C, in order to confirm that ZFPL1 protein is expressed in prostate cancer cell lines and the expressed protein in the prostate is of the same size as in other organs, the presence of ZFPL1 protein in PC3-CTR cell lysates was investigated. ZFPL1 immunoprecipitates were obtained, and its molecular weight was determined by western blot analysis. ZFPL1 immunoprecipitates displayed a band of ~35 Kda, which is consistent with the reported size of 34.1 kDa. For all of the FIG. 1 graphs, the * symbol indicates P<0.05 and the ** symbol indicates P<0.0001 (significantly different from the control, ordinary One-Way ANOVA and Tukey's multiple comparison test).

Regulation of ZFPL1 mRNA expression by CT and Testosterone: Turning next to FIGS. 1D and 1E, to confirm that ZFPL1 is a CT-inducible gene, the effect of CT on ZFPL1 mRNA abundance was examined in PC3-CTR and LNCaP-C4 cell lines. The cells were cultured overnight and treated with CT (1-100 nM) for 4 hours. RNA was extracted and reverse transcribed, and qRT-PCR for ZFPL1 was performed. The bar graphs represent the mean relative ZFPL1 mRNA abundance±SEM (n=3) in LNCaP-C4 (FIG. 1D) and PC3-CTR (FIG. 1E) cells after treatment with CT at 0, 5, 10, 50 and 100 nM concentrations. The results show that CT induced dose-dependent increases in ZFPL1 mRNA levels in both cell lines. The control was set as 1.0.

Because testosterone is the primary hormone for structural and functional integrity of the prostate, its effect on ZFPL1 expression was also examined. The same procedures were used as for the CT testing, except the cells were treated with testosterone agonist R1881 (10 mM-10 nM) overnight. FIG. 1F shows that androgen receptor agonist R1881 induced a similar dose-dependent increase in ZFPL1 mRNA expression in LNCaP-C4 cell line (mean±SEM of n=3). The same was not investigated in PC3-CTR cells as they lack androgen receptors. The control was set as 1.0. For all of the FIG. 1 graphs, the * symbol indicates P<0.05 and the ** symbol indicates P<0.0001 (significantly different from the control, ordinary One-Way ANOVA and Tukey's multiple comparison test).

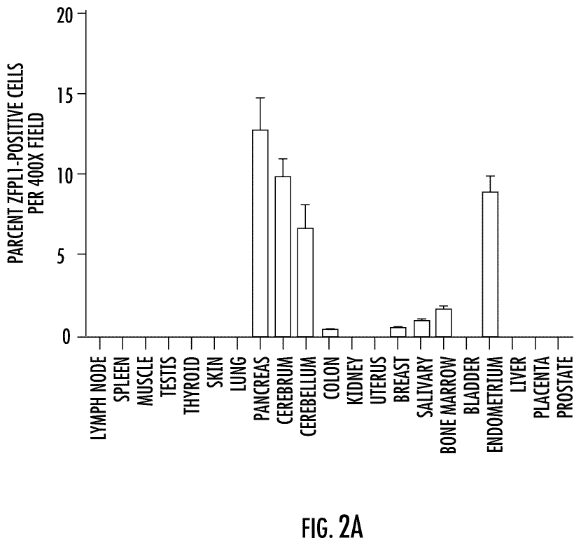

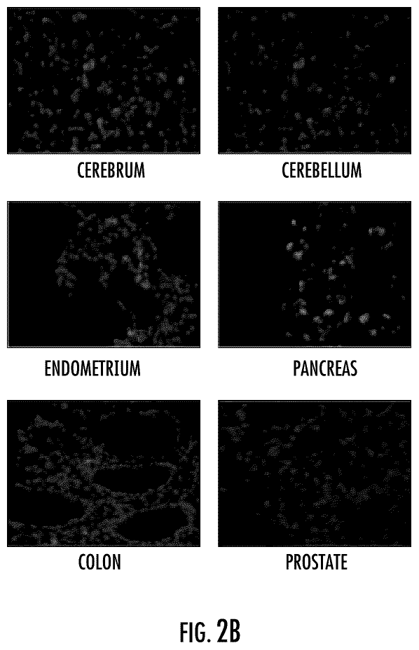

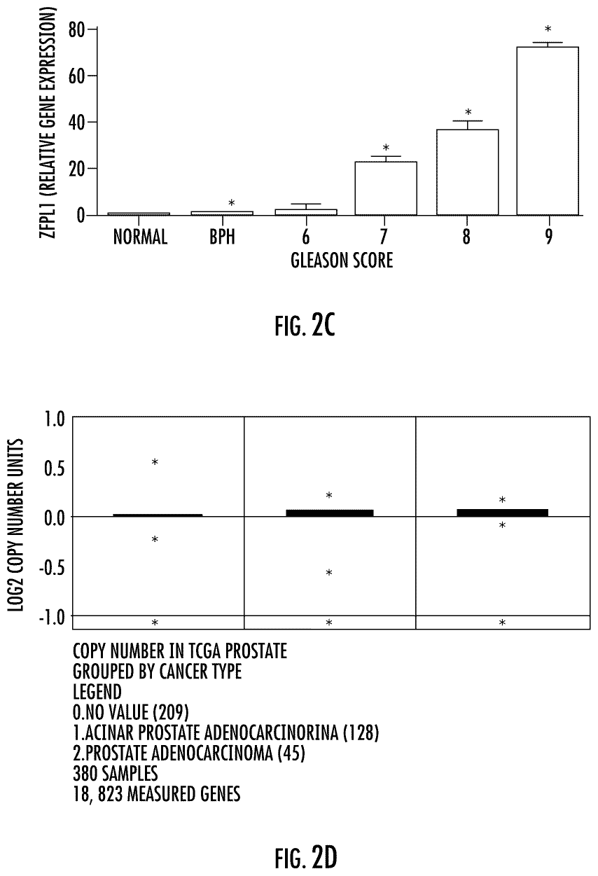

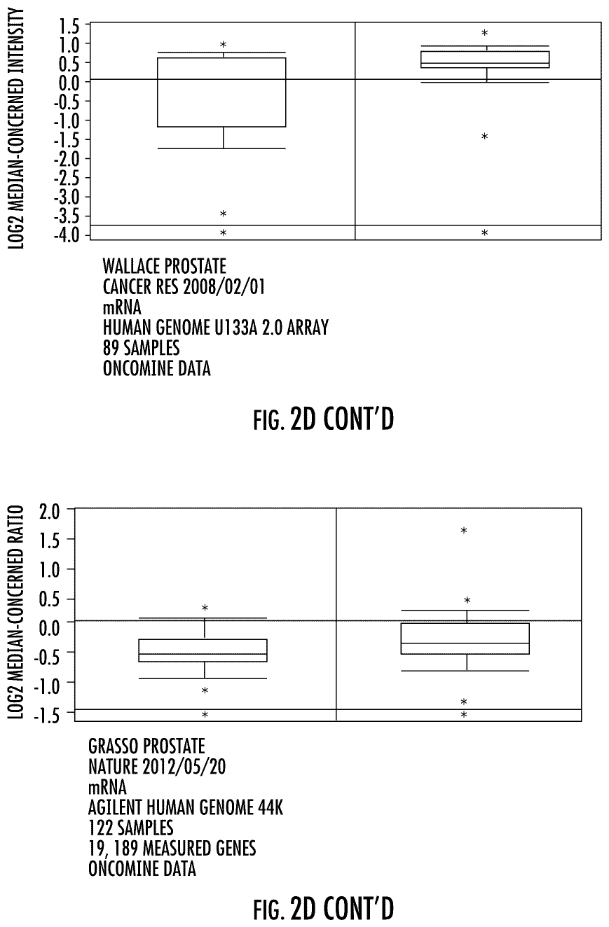

Expression of ZFPL1 in normal human tissues: ZFPL1 immunofluorescence was performed on TRP-1 microarray containing sections of normal human tissues. The percentage of positive ZFLP1-immunopositive cells per field (magnification ×400) were counted. The mean±SEM (n=6) percentage of ZFPL1 immunopositive cell populations per field is graphed in FIG. 2A. The * symbol indicates P<0.05 (significantly different from the normal prostate, ordinary One-Way ANOVA and Tukey's multiple comparison test). The results show that ZFPL1 protein was expressed in cell populations of cerebrum, cerebellum, pancreas and endometrium. However, no ZFPL1-immunopositive cells were detected in normal human prostate and several other human organs. FIG. 2B includes representative micrographs of ZFPL1-positive cell populations in various ZFPL1-positive organs from the experiment of FIG. 2A.

ZFPL1 mRNA in normal and malignant prostates: To measure ZFPL1 mRNA abundance in normal and pathological prostate tissues, total RNA was extracted from frozen primary prostate specimens and used for RT-qPCR. FIG. 2C is a bar graph that presents the relative ZFPL1 mRNA abundance in normal, BPH, and prostate cancer specimens with different Gleason scores. The * symbol indicates P<0.05 (significantly different from the normal prostate, ordinary One-Way ANOVA and Tukey's multiple comparison test). The results show that ZFPL1 mRNA was barely detectable in normal prostates and its levels increased slightly in BPH. However, the increase in ZFPL1 mRNA levels was remarkably higher and statistically significant in prostate cancer specimens. Moreover, the mRNA abundance in prostate cancer tissues increased with increase in the Gleason score of prostate cancer tumor specimens. For example, ZFPL1 mRNA abundance in tumors of Gleason score 9 was over 70-fold higher than that in a normal prostate. These results suggest that the aggressiveness of a tumor can be predicted by the serum ZFPL1 levels of the patient. Since the Gleason score assessment can only be made by examining the biopsy of the patient, the ZFPL1 test can provide a non-invasive alternative for assessing the aggressiveness of the cancer. In FIG. 2D, the data of public portals such as TCGA and Oncomine also revealed an increased expression of ZFPL1 in prostate cancer tissues compared with normal prostate tissues. The * symbol indicates P<0.05 (significantly different from the normal prostate, ordinary One-Way ANOVA and Tukey's multiple comparison test).

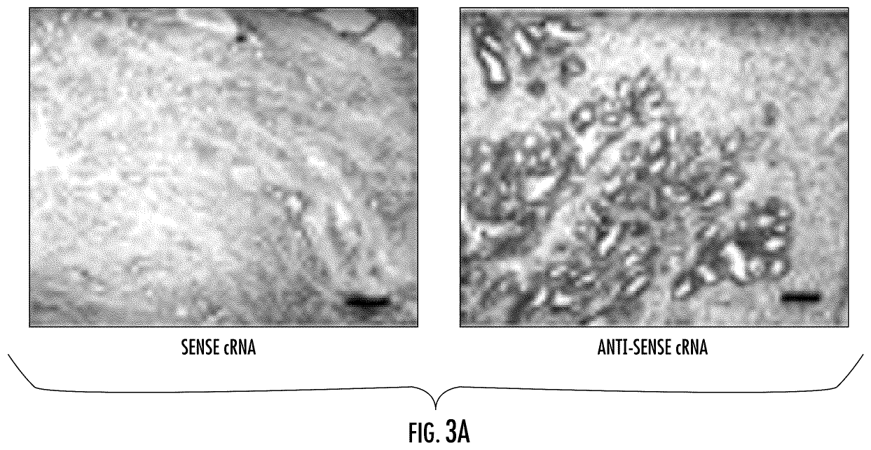

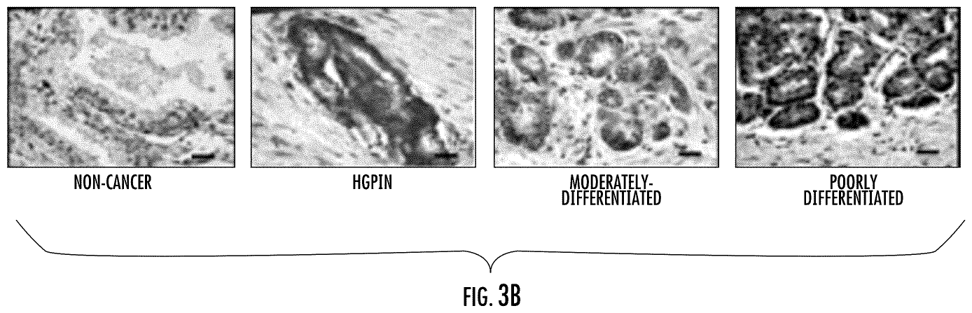

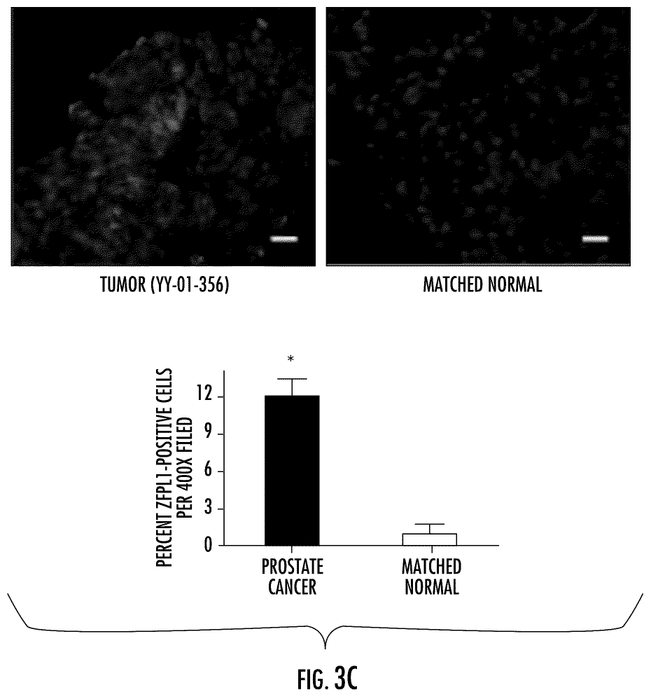

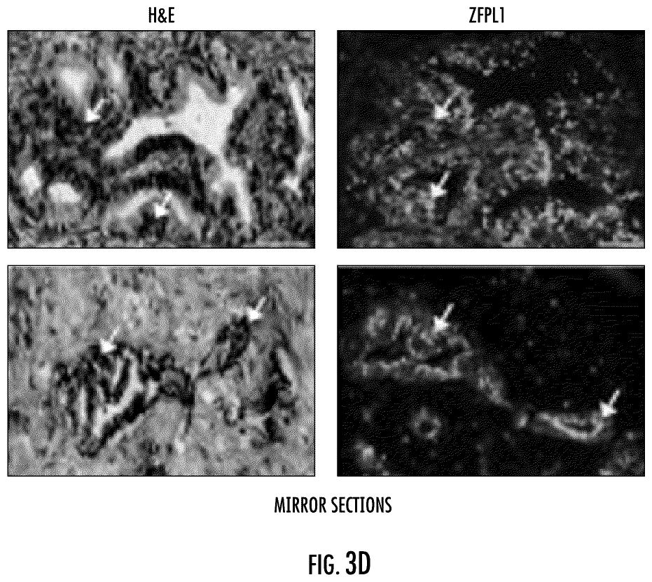

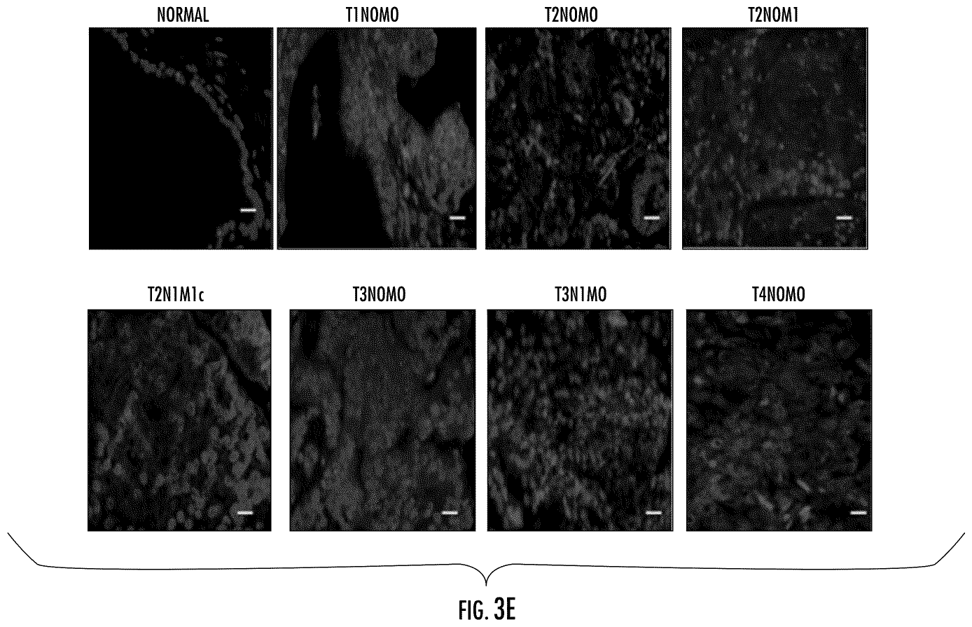

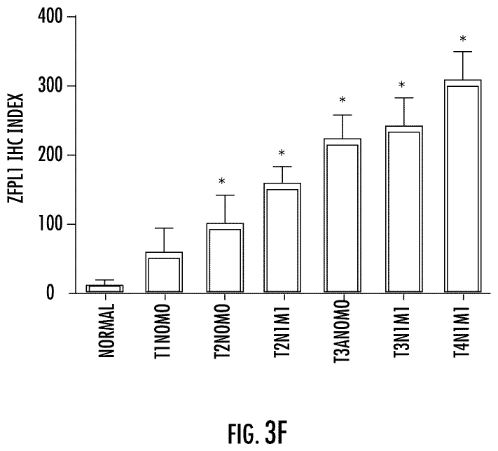

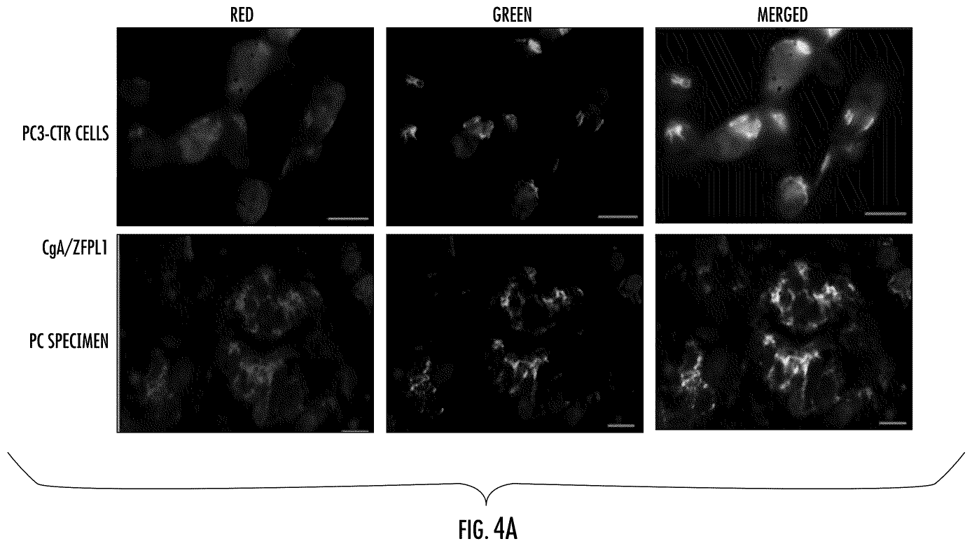

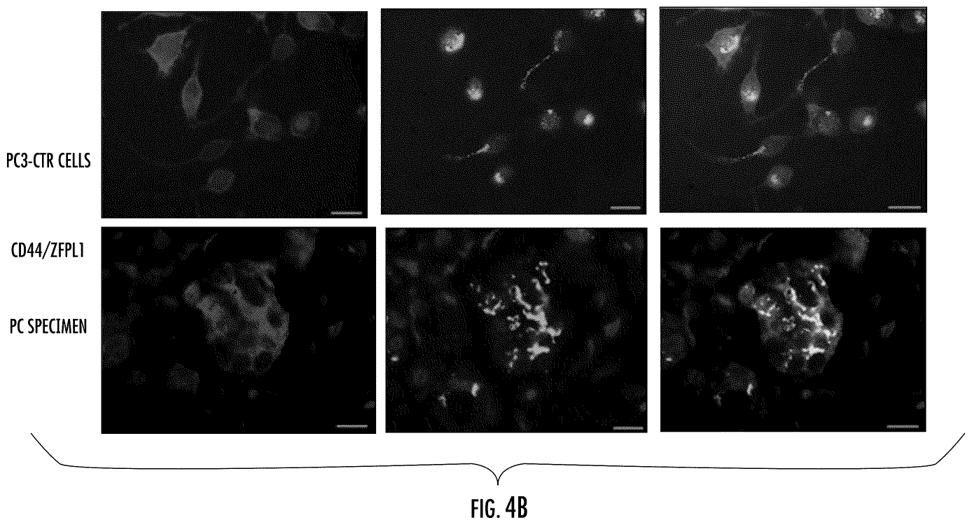

ZFPL1 mRNA expression in clinical prostate specimens: ZFPL1 mRNA was also examined in several paraffin-embedded human prostate specimens by in situ hybridization (ISH) using digoxigenin 11-UTP-labeled ZFPL1 sense (non-specific binding) and anti-sense (specific binding) riboprobes. The specificity of the ISH method is demonstrated in the photomicrographs of FIG. 3A. (Scale bar=100 μm). It was shown that only antisense ZFPL1 siRNA (FIG. 3A, right panel), but not sense ZFPL1 siRNA (FIG. 3A, left panel), hybridized with endogenous ZFPL1 mRNA in a prostate cancer specimen.

This technique was then applied to 78 prostate sections, which varied from BPH, high grade prostate intraepithelial neoplasia (HGPIN), and prostate cancers with Gleason scores between 1-6 and 7-10. The processed sections were then observed under Nikon Optiphot microscope, and six or more digital micrographs per section were captured. Representative photomicrographs from this experiment are shown in FIG. 3B. (Scale bar=50 μm). The staining in digital micrographs (×400) was quantitated by determining the area of staining using iImage Biovision image analysis program. The intensity of the staining was determined in the scale of 0-3 (0 for none, 1 for low, 2 for intermediate and 3 for high). The IHC index was calculated by multiplying the area of staining with the scale of staining. As is visible from the images in FIG. 3B, ZFPL1 transcript was undetectable in benign specimens, was detected in HGPIN specimens and significantly increased with tumor progression. Quantitated data presented in Table 2 demonstrates the lowest value for benign acini, with a significant increase in HGPIN, and even more remarkable increase in prostate cancer specimens with higher Gleason scores.

TABLE 2

| | | | PC | PC |
|---|---|---|---|---|
| | Benign | HGPIN | (Gleason: | (Gleason: |
| | acini | acini | 1-6) | 7-10) |
| Number | 23 | 11 | 21 | 23 |
| of cases | | | | |
| Mean ± | 13.51 ± 3.01 | 39.73 ± 3.17* | 47.13 ± 4.95^{a} | 93.25 ± 3.83^{b} |
| SEM | | | | |

^{a}P < 0.05 represents groups significantly different than benign acini group;
^{b}P < 0.05 represents group significantly different than rest of the groups. PC, prostate cancer.
*p < 0.05 (significantly different from benign acini, unpaired t-test).

Expression of ZFPL1 in prostate tumors: immunohistochemistry: FIG. 3C compares ZFPL1 immunofluorescence in prostate tumors (left) to matched normal tissues (right). ZFPL1 protein expression (red) was cancer-specific, and no staining was detected in a matched normal tissue. The nuclear stain is DAPI (blue). (Scale bar=50 μm). FIG. 3C also includes a bar graph that represents the mean percentage (n=6) of ZFPL1 immunopositive cells per field (magnification, ×400) in the prostate cancer tissue vs matched normal tissue test. The * symbol indicates P<0.0001 (paired t-test). In a total of ~12% of tumor cells, ZFPL1 protein was detected in cancer tissue with no positivity in a matched normal tissue.

Localization of ZFPL1 in cancer tissue: To investigate whether ZFLP1 was localized to histologically positive cancer area of the specimen, H&E and ZFPL1 immunofluorescence was performed in serial sections of the same biopsy specimens. In FIG. 3D, the representative photomicrographs on the left show H&E staining of human prostate cancer tissue sample (dark bluish staining is for nuclear hematoxylin and pink staining is for eosin) while the photomicrographs on the right show green immunofluorescent labeling for ZFPL1 and blue color labeling for nuclear DAPI. (Scale bar=50 μm). As is demonstrated by the white arrows in FIG. 3D, ZFLP1 staining was selectively localized in the cancerous part of the specimen (as indicated by hematoxylin-stained large nuclei).

ZFPL1 immunoreactive cell populations in prostate cancer increases with tumor progression: Tumor stage-specific expression of ZFPL1 protein was examined by immunofluorescence of US Biomax prostate cancer tissue microarray. The array contained sections of 80 specimens (73 PCs and 7 normal). The immunohistochemistry (IHC) was performed, and multiple fluorescent images of each specimen were captured. The number of ZFPL1-immunopositive cells (red-TRITC) and total cells (blue-DAPI) per field (magnification, ×400) were counted and IHC Index was determined as aforementioned. FIG. 3E is representative photomicrographs that reveal ZFPL1-immunopsitive cells (red) and nuclear DAPI (blue) in the different samples of the prostate cancer tissue microarray. (Scale bar=50 μm). As is visible in FIG. 3E, ZFPL1 immunostaining was distributed in the cytoplasm of cells in epithelia of prostate tumors but not in epithelia of normal prostate. Moreover, an apparent increase in the number of immunopositive cells as well as in the staining intensity was observed with increases in tumor stage. The bar graph in FIG. 3F presents the quantitated data of the prostate cancer tissue microarray of FIG. 3E. The mean±SEM (n=6) IHC index of each specimen in the microarray was calculated and plotted against the stage of PC. The mean IHC index of each cancer group except TIN0MO was significantly different from the control. The * symbol indicates P<0.005 (One Way ANOVA and Tukey's multiple comparison test). These results suggest that the IHC index of PC specimens increased with increase in tumor stage and was highest in metastatic tumors of stage T4N1M1.

ZFLP1 co-localizes with chromogranin A (a neuroendocrine marker) and CD44 (a cancer stem cell marker): Fixed PC3-CTR cells and sections of paraffin-embedded prostate cancer specimens were processed for double immunofluorescence using pairs of primary antibodies against ZFLP1+CgA or ZFPL1+CD44. In FIG. 4A, the representative photomicrographs show the colocalization of ZFPL1 and chromogranin A (CgA) in PC3-CTR cells (upper panels) and human prostate cancer tissue (lower panels). (Scale bar=50 μm). The results illustrate that in cells as well as tissues, ZFPL1 (green) co-localized with CgA (red) in same cells. Similarly, as shown in the representative immunofluorescence photomicrographs of FIG. 4B, ZFPL1 (green) co-localized with CD44 (red) in PC3-CTR cells (upper panels) and human prostate cancer tissue (lower panels). (Scale bar=50 μm). iVision image analysis program statistically evaluated co-localization of both fluorescent dyes in each digital image and calculated Pearson's co-efficient (maximum being 1.000). CgA-ZFPL1 and CD44-ZFPL1 co-localization data showed a Pearson's co-efficient value of >0.83 and >0.8 (mean value) respectively, suggesting a very strong co-localization of these three antigens in same cells.

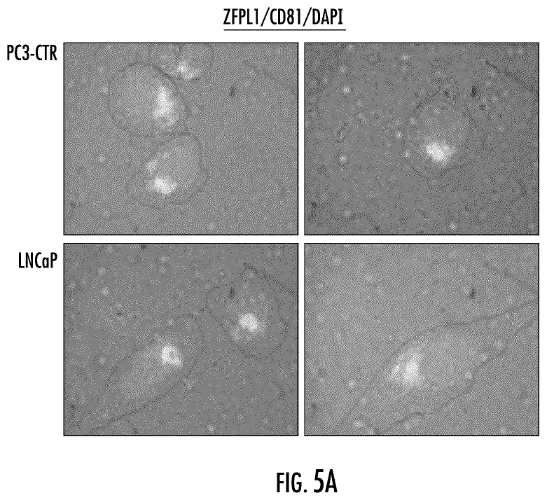

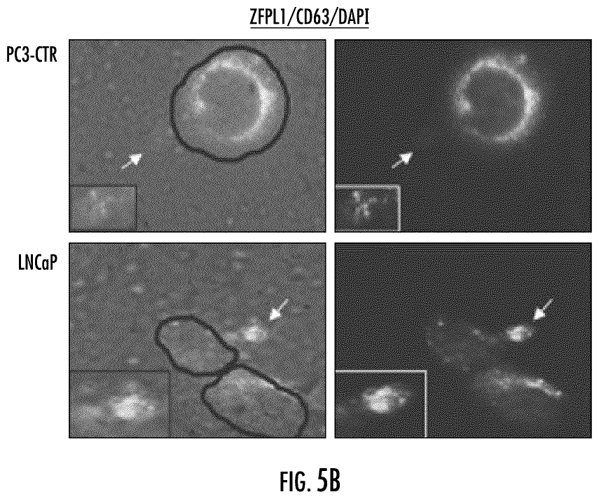

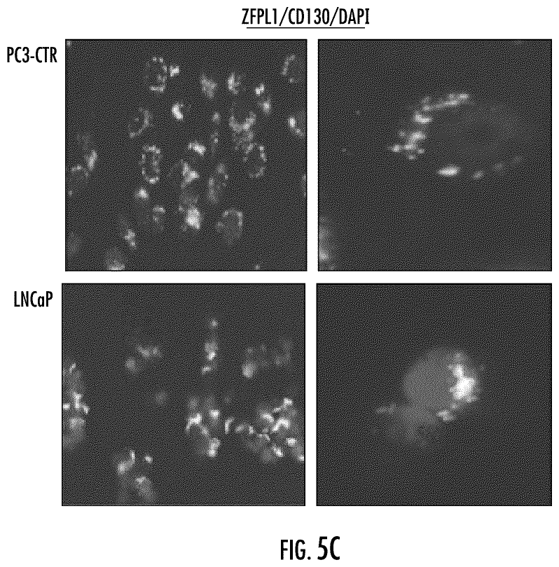

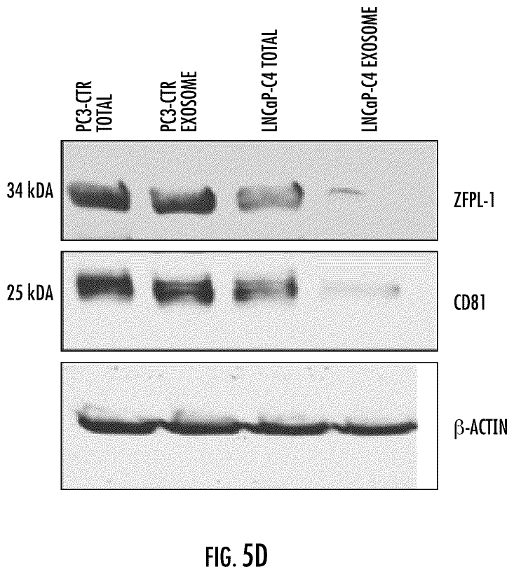

Subcellular localization of ZFPL1 protein in cultured PC cells: In cultured PC3-CTR and LNCaP-C4 cells, the subcellular localization of ZFPL1 (green) was examined by triple immunofluorescence using markers of the Golgi body GM130 (red), exosome CD81 (red), exosome-secretosome CD63 (red), and counterstaining for nucleus DAPI (blue). FIG. 5A is representative photomicrographs that show the colocalization of ZFPL1 and exosome CD81 in PC3-CTR and LNCaP PC cells. FIG. 5B is representative photomicrographs that reveal the colocalization of ZFPL1 and exosome/secretosome maker CD63. Cell borders were traced to show the location of exosomes with respect to a cell. Inset showed the magnified image (magnification, ×1,000) of the location pointed by the arrow. FIG. 5C is representative photomicrographs that show the colocalization of ZFPL1 and Golgi body marker GM130. (Scale bars=25 μm). Co-localization of ZFPL1 with CD81 and CD63 in FIGS. 5A and 5B suggested that ZFPL1 may be an exosomal protein. Moreover, the co-localization of ZFPL1 with GM130 in FIG. 5C suggested its presence in Golgi.

In FIG. 5D, the presence of ZFPL1 in exosomes was confirmed by isolating the exosomal fraction of PC3-CTR cells and LNCaP-C4 cells and confirming its presence in the isolate by western blot analysis. 8-actin is the loading control. Co-precipitation of ZFPL1 with CD81 (exosome marker) in exosome isolate confirms the presence of ZFPL1 in the exosomes of prostate cancer cell lines. Notably, relative presence of ZFPL1 immunoreactivity in PC3-CTR cells was markedly higher than LNCaP-C4 cells.

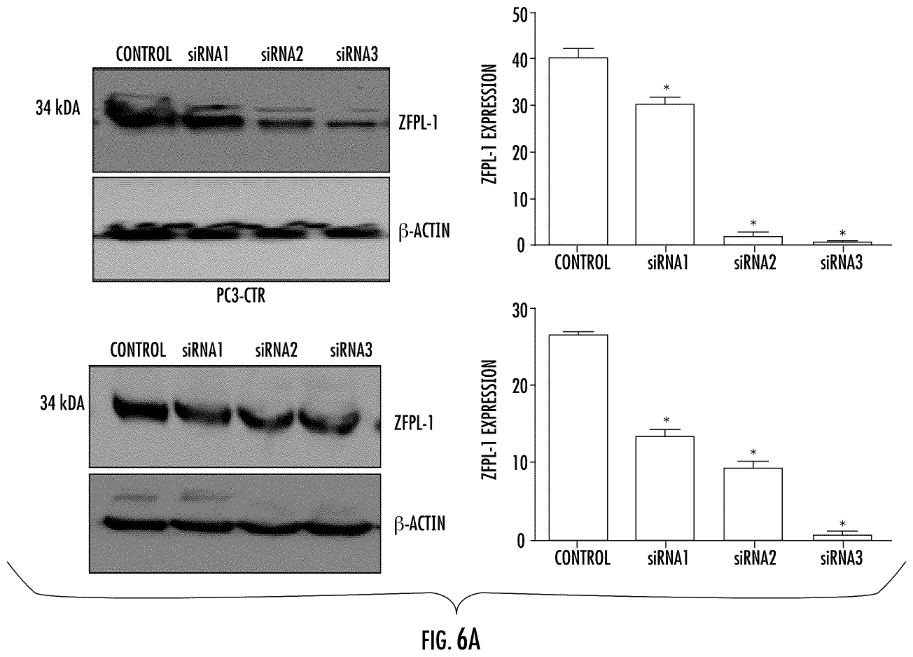

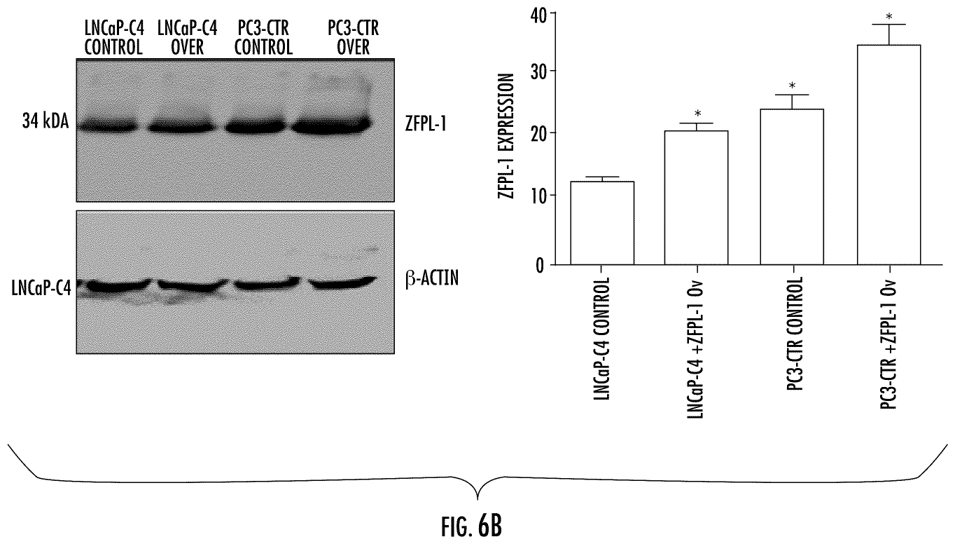

Function of ZFPL1 in prostate cancer cells: To identify the potential role of ZFPL1 in prostate cancer progression, the effect of ZFPL1 knockdown and overexpression on prostate cancer cell characteristics, such as the rate of cell proliferation, invasion, or apoptosis, was examined. ZFPL1 overexpression was accomplished by transfecting constitutively active ZFPL1 expression plasmid. The knockdown was accomplished by transfection of either of 3 ZFPL1 siRNAs. β-actin was used as a housekeeping control. The knockdown (FIG. 6A) and overexpression (FIG. 6B) were verified using western blotting and protein bands were quantified by densitometry. The * symbol indicates P<0.05. The results in FIG. 6A show that siRNA1 appeared to be least potent in attenuating ZFPL1 expression, whereas siRNA3 appeared to be the most potent and was used in subsequent experiments unless specifically stated otherwise. The results in FIG. 6B show that the transfection of ZFPL1 expression plasmid in PC3-CTR and LNCaP-C4 cells led to an increase in ZFPL1 protein levels in both cell lines.

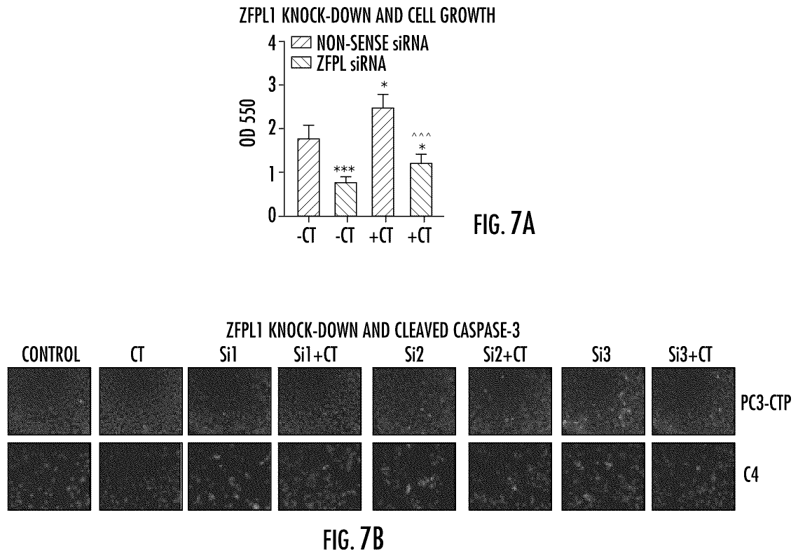

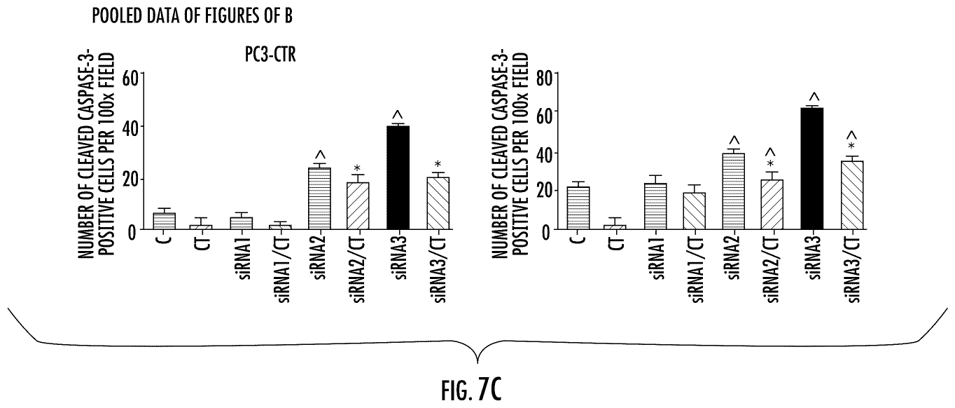

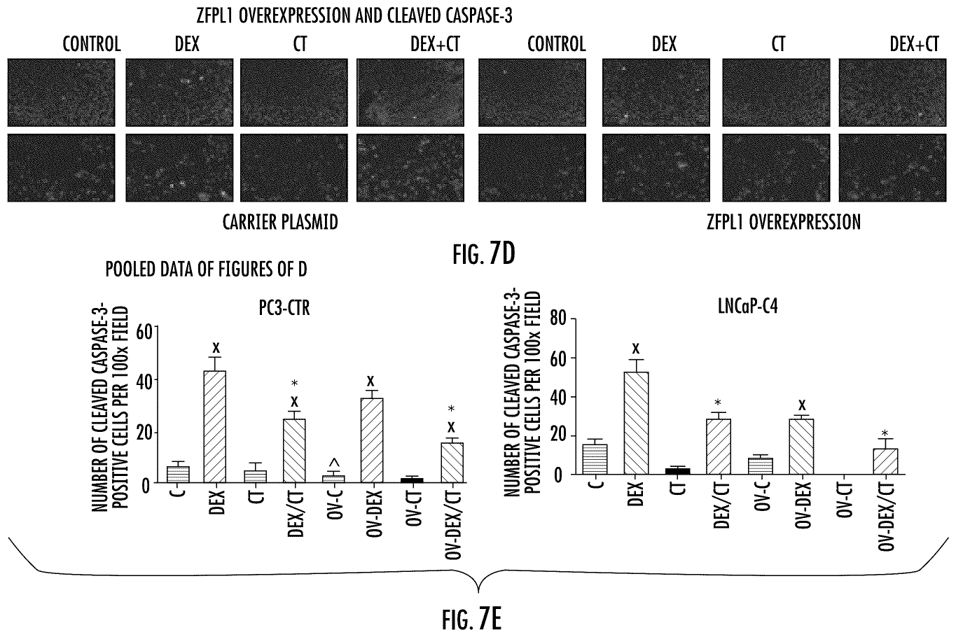

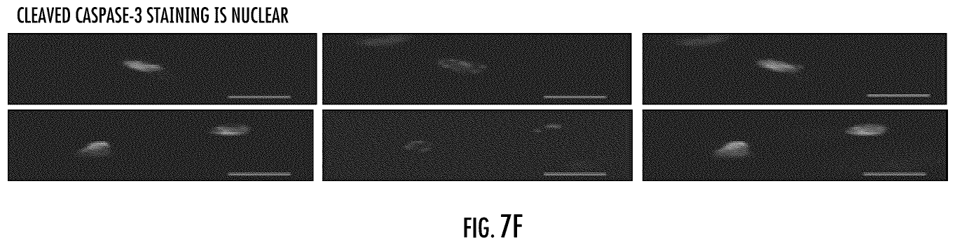

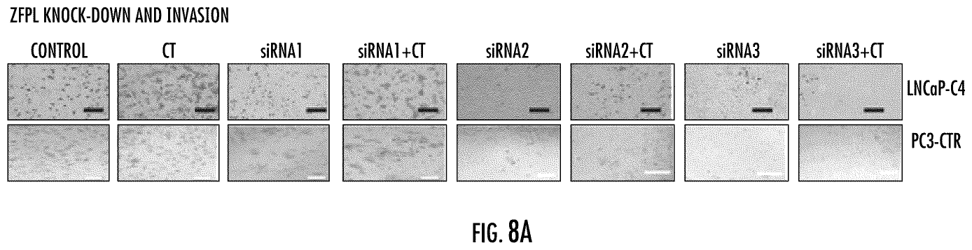

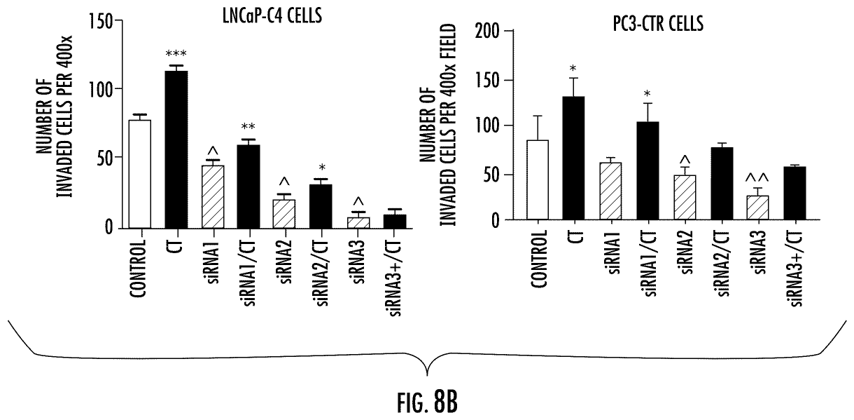

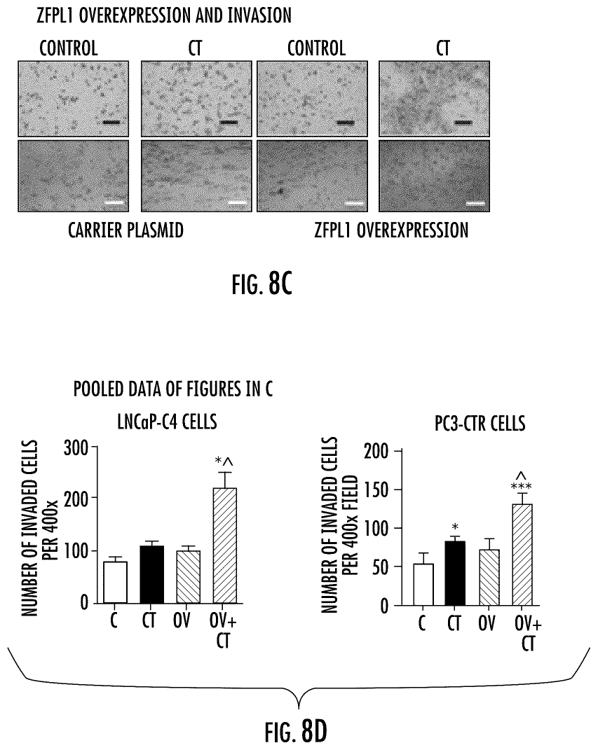

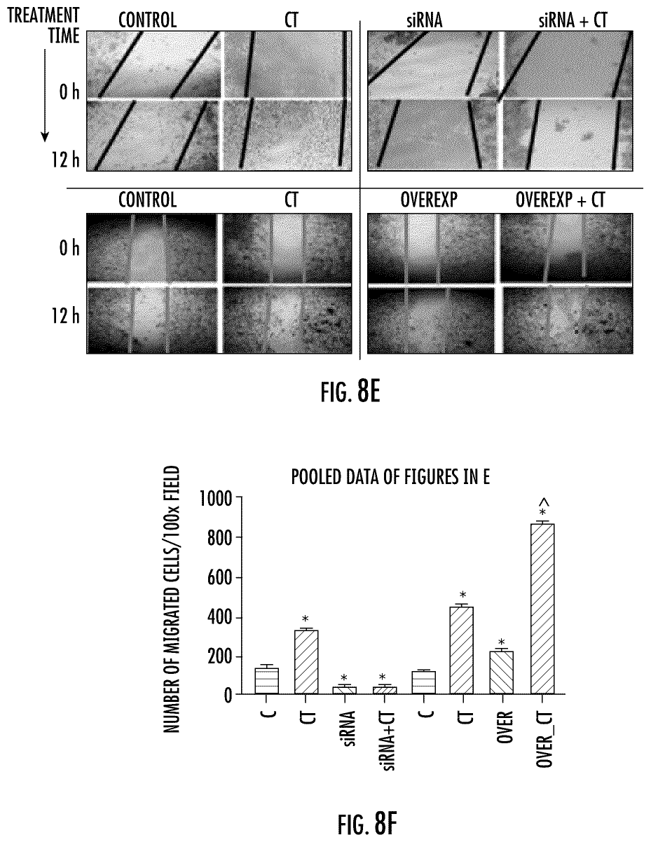

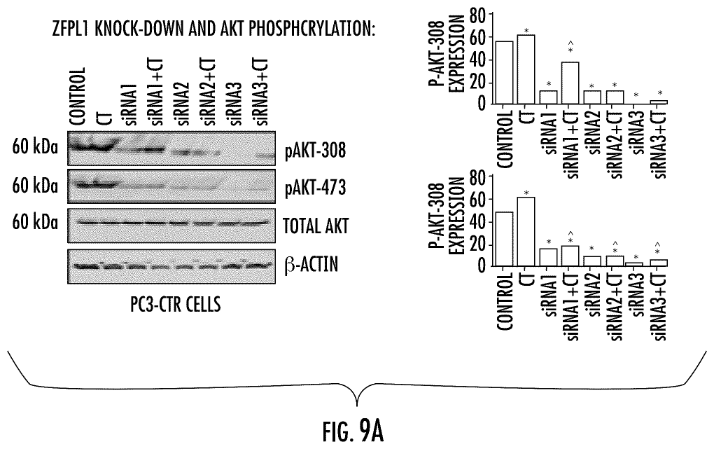

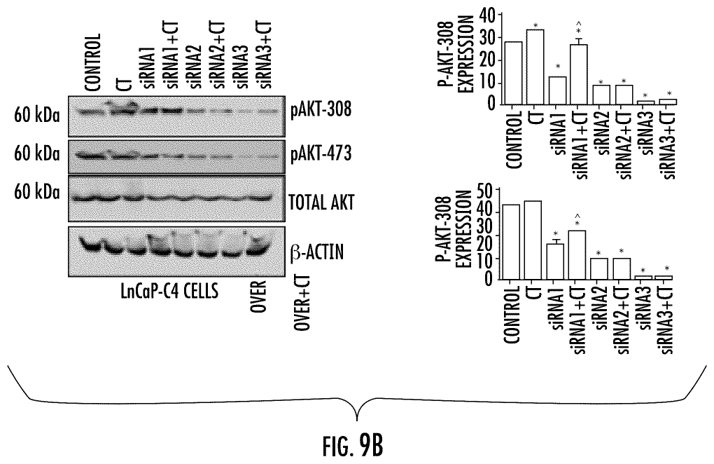

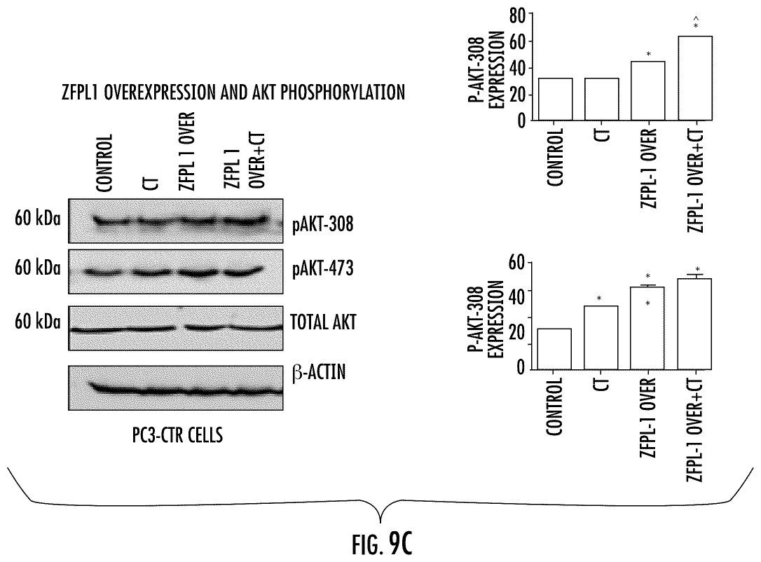

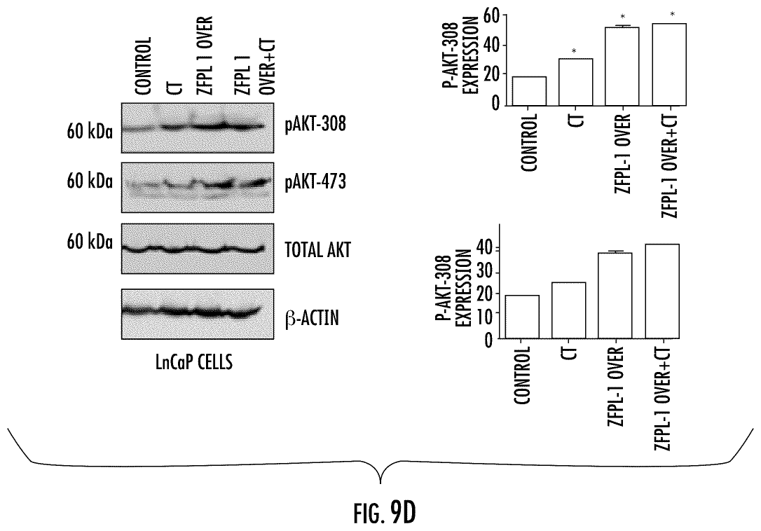

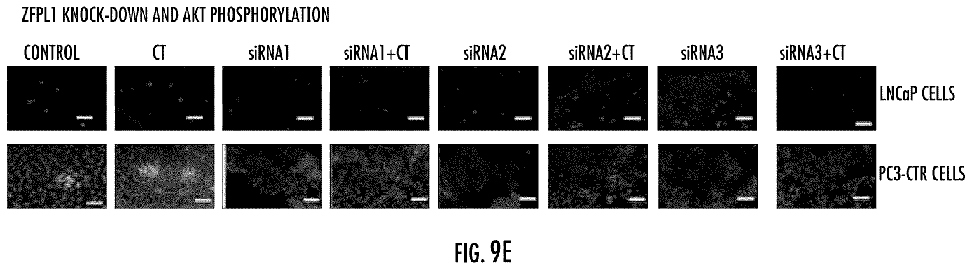

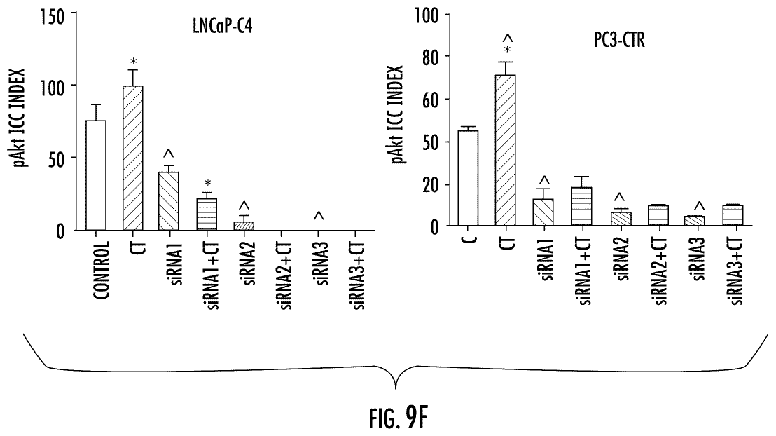

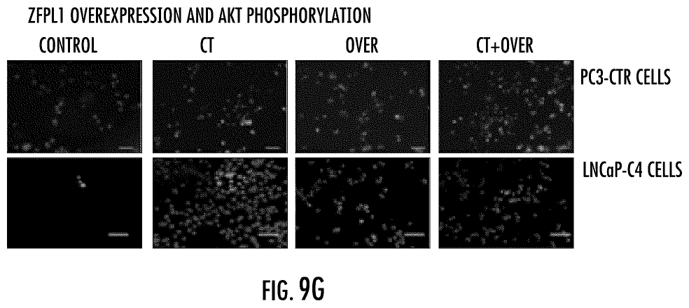

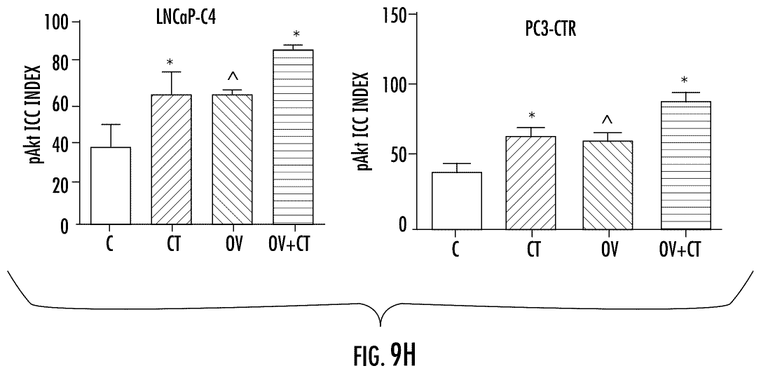

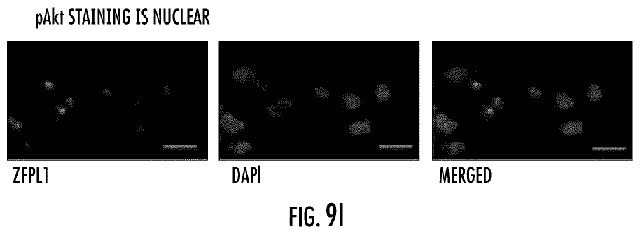

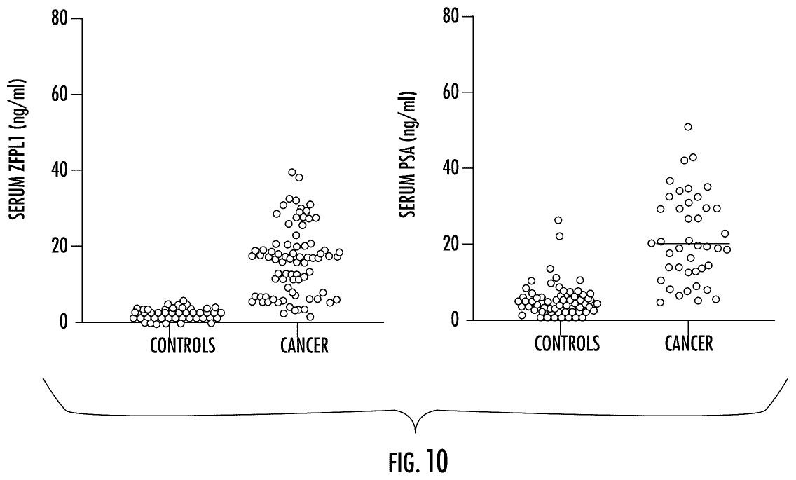

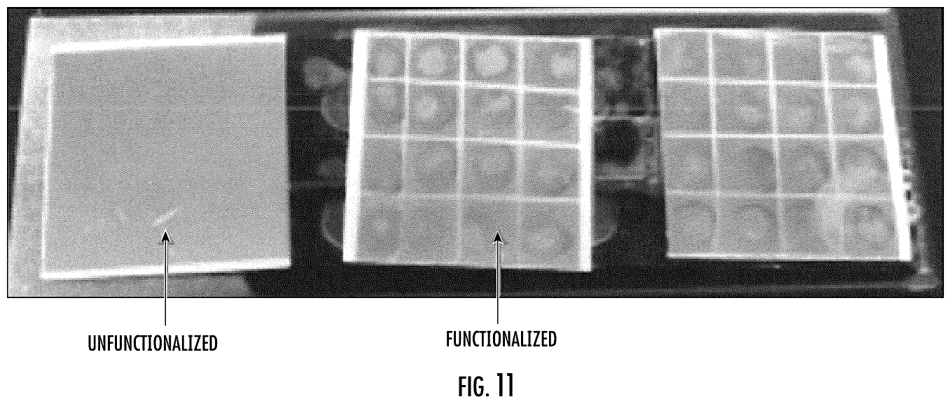

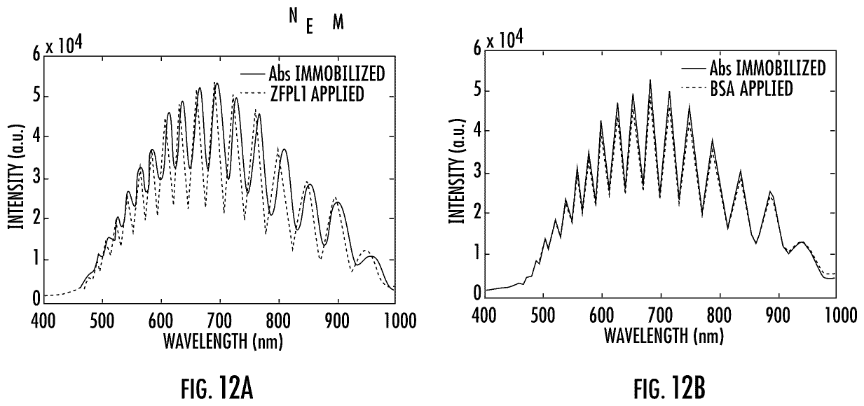

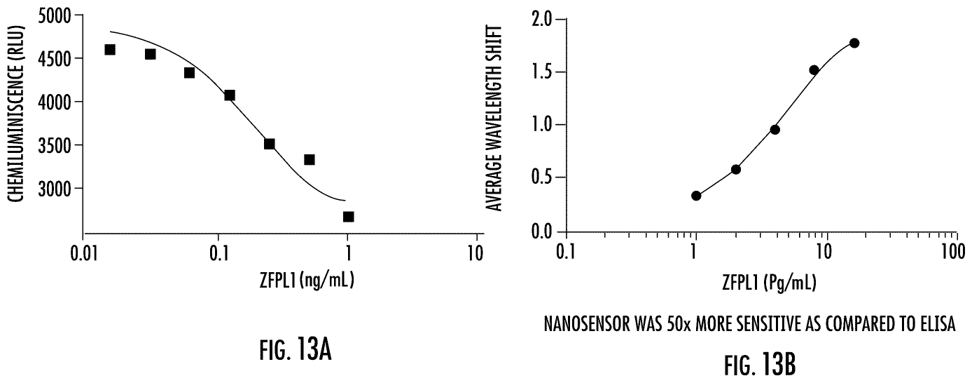

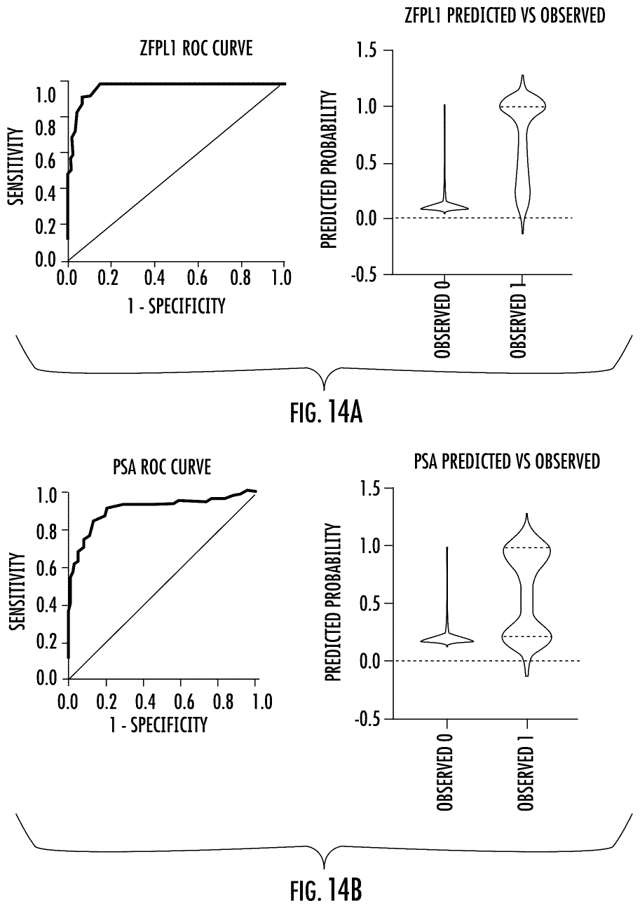

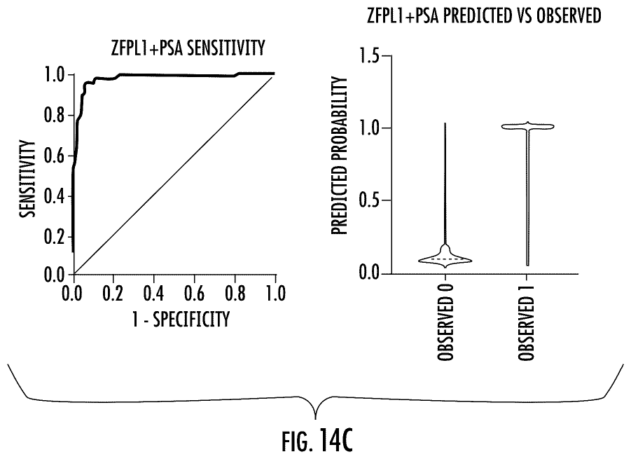

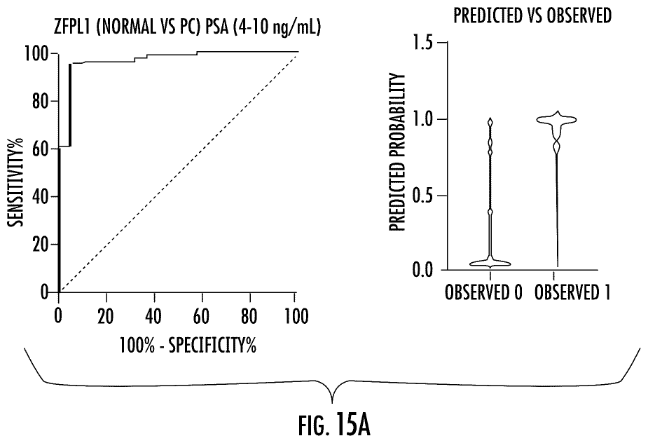

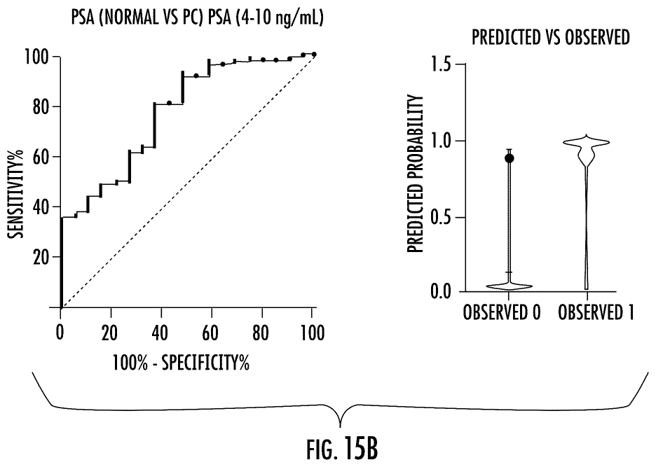

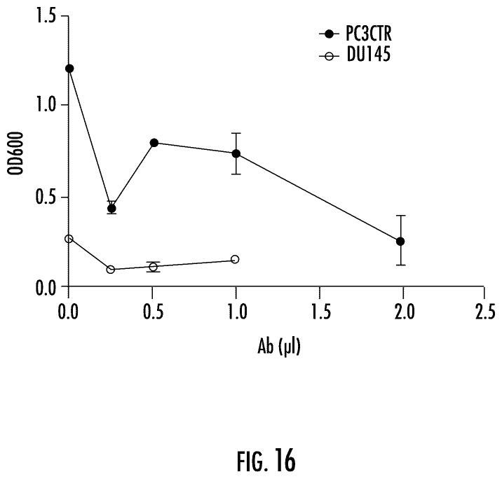

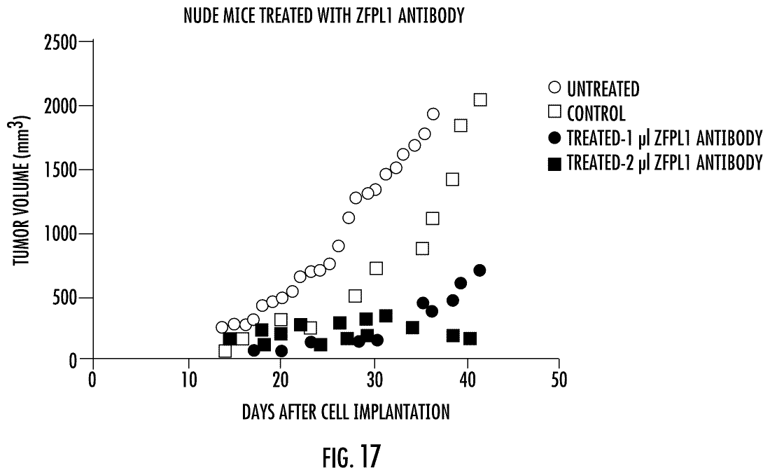

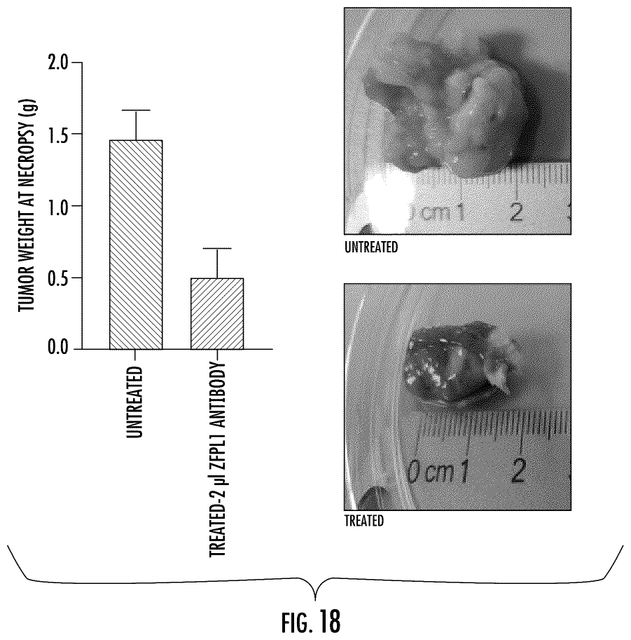

Effect of ZFPL1 knockdown on prostate cancer cell proliferation: FIG. 7A is a bar graph showing the effect of ±10 nM CT on proliferation of PC-3CTR cells that received either non-sense siRNA or ZFPL1 siRNA. The data are presented as the mean OD595±SEM (n=4). The * symbol indicates P<0.05 and the *** symbol indicates P<0.0001 compared to the control receiving non-sense siRNA (unpaired t-test). The \hat{ }\hat{ }\hat{ } symbol indicates P<0.0001 compared to +CT receiving non-sense siRNA (unpaired t-test). The results show that the knockdown of ZFPL1 in PC3-CTR cells led to a significant decrease in basal and CT-stimulated cell proliferation.

Effect of ZFPL1 knockdown and overexpression on apoptosis of prostate cancer cells: Apoptosis in PC3-CTR and LNCaP cells was examined by analyzing the presence of cleaved caspase-3 in the nucleus by immunofluorescence. The representative photomicrographs in FIG. 7B demonstrate the effect of either non-sense (control) or ZFPL1 siRNA (1, 2 or 3)±CT on cleaved caspase 3 expression in PC3-CTR (upper panel) and LNCaP-C4 (lower panel) cells. The blue color of the DAPI stain shows the nucleus (Scale bar=100 μm). The results demonstrate that knockdown of ZFPL1 led to a visible increase in cleaved caspase-3-positive PC3-CTR cells. However, CT could reverse/reduce this effect significantly. The pooled data from these experiments is presented in FIG. 7C. The graph presents the number of cleaved caspase 3-positive cells per field (magnification, ×400) against ±CT treatment. The * symbol indicates P<0.05 and the ** symbol indicates P<0.001 compared to +CT of its own group. The \hat{ } symbol indicates P<0.05 compared to the corresponding non-sense siRNA control (One way ANOVA and Tukey's multiple comparison test). The results suggested that the knockdown of ZFPL1 by siRNAs 2 and 3 led to a significant increase in the number of cleaved caspase 3-positive cells in both cell lines and that CT could reverse/reduce this effect.

The effect of ZFPL1 overexpression on DEX-induced apoptosis was examined after treating cells with/without DEX. In FIG. 7D, the first four pairs of photomicrographs show the expression of cleaved caspase 3 (green) in PC3-CTR and LNCaP-C4 cells expressing carrier plasmid. The next four pairs of photomicrographs reveal the expression of cleaved caspase 3 in cells overexpressing ZFPL1. The cells also received either vehicle, DEX (10 μM), CT (10 nM) or DEX+CT. DAPI stain is shown in blue. (Scale bar=100 μm). Again, the results clearly revealed that either the treatment with CT and/or ZFPL1 overexpression significantly attenuated DEX-induced apoptosis in both cell lines. FIG. 7E illustrates the pooled quantitative data of these experiments. The mean number±SEM of cleaved caspase 3-labeled cells per field (magnification, ×400) were plotted against the treatment+CT±DEX. The * symbol indicates P<0.05 compared to DEX+CT; the X symbol indicates P<0.001 compared to ZFPL1-overexpression (One way ANOVA and Tukey's multiple comparison test). The \hat{ } symbol indicates P<0.05 compared to C (ordinary one-way ANOVA and Tukey's multiple comparison test). The results show that ZFPL1 overexpression and/or treatment with CT significantly reduced apoptotic populations in both cell lines. In FIG. 7F, representative photomicrographs show the localization of cleaved caspase-3 staining in the nuclei of LNCaP-C4 cells. (Scale bar=25 μm). These results show that the cleaved caspase-3 staining in LNCaP-C4 cells was nuclear.

Effect of ZFPL1 knockdown and overexpression on invasion of prostate cancer cells: The representative photomicrographs in FIG. 8A show the effect of +10 nM CT on invasiveness of PC3-CTR cells receiving either non-sense siRNA or ZFPL1 siRNA (1, 2 or 3) (Scale bar=50 μm). The knockdown of ZFPL1 significantly decreased basal and CT-induced invasion of LNCaP-C4 and PC3-CTR cells. The bar graphs in FIG. 8B reveal the pooled data of these invasion assays presented as the mean±SEM number of invading cells per field (magnification, ×400) with PC3-CTR and LNCaP-C4 cells receiving either non-sense siRNA, siRNA1, siRNA2 or siRNA 3. The * symbol indicates P<0.05, the ** symbol indicates P<0.001, and the *** symbol indicates P<0.0001 comparing-CT to +CT in each group. The \hat{ } symbol indicates P<0.01 and the \hat{ }\hat{ } symbol indicates P<0.001 comparing non-sense siRNA to ZFPL1 siRNA. All of the above P-values were calculated using one-way ANOVA and Tukey's multiple comparison test.

In FIG. 8C, the representative photomicrographs reveal the effect of +10 nM CT on invasiveness of LNCaP-C4 and PC3-CTR cells expressing either carrier pCMV5-XL4 plasmid or the plasmid with ZFPL1 expression plasmid. The results show that the overexpression of ZFPL1 in either cell line led to an increase in basal and CT-induced invasion. The bar graphs in FIG. 8D show pooled data (mean±SEM) of four separate invasion assays with PC3-CTR and LNCaP-C4 cells, respectively. The * symbol indicates P<0.05, the symbol indicates P<0.001, and the *** symbol indicates P<0.0001 comparing −CT to +CT in each group. The \hat{ } symbol indicates P<0.05 comparing CT to OV+CT. All of the above P-values were calculated using one-way ANOVA and Tukey's multiple comparison test.

A similar study was also conducted to examine cell migration of PC3-CTR cells in a wound-healing assay. The photomicrographs of the top left quadrant of FIG. 8E revealed the wound of PC3-CTR cell layer at 0 h and after 12 h in the absence or the presence of 10 nM CT. The top right quadrant of FIG. 8E shows similar experiments with PC3-CTR cells with ZFPL1 knocked down using siRNA3. The results are demonstrated in the bar graph of FIG. 8F, presented as the mean±SEM of the number of migratory cells migrated in a wound (magnification, ×100) in the four separate wound healing assays. This pooled data shows that CT promoted the cell migration of PC3-CTR cells. However, when ZFPL1 was knocked down, the baseline cell migration was reduced, and CT also failed to promote cell migration.

The next experiment examined the effect of ZFPL1 overexpression in PC3-CTR cells. The photomicrographs of the bottom left quadrant of FIG. 8E again showed that CT promotes cell migration in PC3-CTR cells. However, as is shown in the bottom right quadrant of FIG. 8E, ZFPL1 overexpression increased cell migration in the absence as well as the presence of CT. The pooled data in FIG. 8F also demonstrates that ZFPL1 overexpression increased cell migration of PC3-CTR cells, and the addition of CT increased it even more. These results were consistent with the effect of ZFPL1 on prostate cell invasion. The * symbol indicates P<0.05 as compared to the control for each group (i.e., either siRNA or overexp). The \hat{ } symbol indicates P<0.05 for overexp compared to overexp+CT (One Way ANOVA and Tukey's multiple comparison test).

ZFPL1 and Akt phosphorylation: Since the knockdown of ZFPL1 led to apoptosis of prostate cancer cells and its overexpression decreased DEX-induced apoptosis, the effect of ZFPL1 on the activation of PI3K survival pathway was investigated by examining phosphorylation of Akt in PC3-CTR and LNCaP-C4 cells. FIGS. 9A and 9B summarize the immunoblot results of the effects of ±10 nM CT on p-Akt473 and p-Akt308 proteins in PC3-CTR (FIG. 9A) and LNCaP-C4 (FIG. 9B) cells receiving either non-sense (control) siRNA or ZFPL1 siRNA1, ZFPL siRNA2, or ZFPL1 siRNA3. Total Akt was used as a control protein, and β-actin was used as the loading control. Also included in FIGS. 9A and 9B are the normalized densitometric bar graphs (p-Akt/total Akt) of the immunoblots. The * symbol indicates P<0.05 and the \hat{ } symbol indicates P<0.05 compared to siRNA+CT (One Way ANOVA and Tukey's multiple comparison test). The data in FIGS. 9A and 9B revealed that the knockdown of ZFPL1 led to a statistically significant decrease in basal and CT-induced phosphorylation of Akt473/Akt308 in both cell lines. CT increased Akt phosphorylation, however, the knockdown of ZFPL1 significantly reduced CT-induced Akt phosphorylation. Consistent with the earlier results, this experiment also revealed siRNA3 to be the most potent in downregulating Akt phosphorylation in both cell lines.

FIGS. 9C and 9D summarize the immunoblot results of the effects of ±10 nM CT on p-Akt473 and p-Akt308 proteins in PC3-CTR (FIG. 9C) and LNCaP-C4 (FIG. 9D) cells transfected with either carrier plasmid or ZFPL1 expression plasmid, respectively. Akt was used as a control protein, and β-actin was used as the loading control. Also included in FIGS. 9C and 9D are the normalized densitometric bar graphs (p-Akt/total Akt) of the immunoblots. The * symbol indicates P<0.05 as compared to the control (One Way ANOVA and Tukey's multiple comparison test). As expected, overexpression of ZFPL1 in these cell lines produced the opposite effect as indicated by a significant increase in basal and CT-induced Akt phosphorylation. The results of this experiment demonstrate that CT induced a minimal increase in Akt473 phosphorylation in LNCaP cells overexpressing ZFPL1, further supporting the possibility that the effect of activation of endogenous CT on PI3K pathway activation in prostate cancer cells is indirect and ZFPL1 may be a key mediator of this CT action.

Phosphorylation of Akt was also observed by immunofluorescence microscopy. The representative photomicrographs of FIG. 9E show changes in phosphorylated (p)-Akt-immunopositive PC3-CTR and LNCaP-C4 cells receiving either non-sense or ZFPL1 siRNA (1, 2 or 3) when treated with ±10 nM CT on pAkt staining (green). The blue color is of DAPI (Scale bar=50 μm). In non-sense siRNA-treated cells, a small population of cells were p-Akt positive (<20%). When treated with 10 nM CT for 30 min, the p-Akt-positive population increased by more than two-fold. When treated with ZFPL1 siRNA, the p-Akt cell population was lower than those treated with non-sense siRNA. However, the treatment with CT increased p-Akt-positive cells but still was markedly less than that in non-sense sRNA treated cells. As is shown in FIG. 9F, the results of the quantified data of these experiments with PC-3CTR and LNCaP-C4 cells suggested that the knockdown of ZFPL1 significantly attenuates/abolishes basal and CT-induced phosphorylation of Akt. The data is presented as the mean±SEM number of p-Akt-immunopositive cells per field (magnification, ×100) of PC3-CTR and LNCaP cells receiving either non-sense siRNA (control) or ZFPL1 siRNAs 1, 2 or 3 in that order. The * symbol indicates P<0.05 comparing the control to the CT-treated cells in each group. The \hat{ } symbol indicates p<0.05 comparing the control to the siRNA-treated cells (One Way ANOVA and Tukey's multiple comparison test).

A similar experiment examined the effect of ZFPL1 overexpression on basal and CT-induced increase of P-Akt in the nuclei of PC-3CTR and LNCaP-C4 cells. The representative photomicrographs of FIG. 9G show the effect of ±10 nM CT on p-Akt-immunopositive cells per field (magnification, ×400; green) in PC3-CTR cells expressing either carrier plasmid or ZFPL-overexpression plasmid. Scale bar=50 μm. P-Akt-positive LNCaP-C4 cells increased by almost 70% when treated with 10 nM CT. A similar increase was identified when LNCaP-C4 cells were transfected with ZFPL1 overexpression vector. When these cells (ZFPLlov) were treated with 10 nM CT, nuclear co-localization of p-Akt increased further by ~35%. As is shown in FIG. 9H, the pooled quantitative data of PC3-CTR and LNCaP-C4 cells suggests that ZFPL1 and CT may have additive effect on Akt phosphorylation. The data is presented as the mean p-Akt ICC Index per field±SEM (magnification, ×100). The * symbol indicates P<0.05 comparing +CT to OV+CT, and the \hat{ } symbol indicates P<0.05 comparing C to OV. (One way ANOVA and Tukey's Multiple comparison test).

Next, it was verified whether pAkt in these cells was localized in the nucleus. In FIG. 9I, representative photomicrographs at higher magnification (×1,000) show the nuclear localization of pAKT (green). Nuclear DAPI is blue (Scale bar=25 μm). It was revealed that P-Akt (green) is co-localized with DAPI at a ×400 magnification.

Generation of monoclonal antibody against ZFPL1: In order to create an antibody to bind with ZFPL1, the ZFPL1 protein was sequenced (SEQ ID NO 11) and its epitopes were determined (see Table 3). In order for the antibody of the present invention or the fragment thereof to specifically bind to the ZFPL1 protein or a variant protein thereof, the antibody specifically binds to a polypeptide within the sequence of the 62nd to the 308th amino acids of the ZFPL1 protein represented by SEQ ID NO: 11, preferably within the range of the 62nd to 77th and 127th to 284th amino acids.

TABLE 3

| No. | Epitope Sequence | Location | SEQ ID NO |
|---|---|---|---|
| 1 | LLLGLLGFLALLALMS | 269-284 | 12 |
| 2 | SRETTRLVCYDLFHWA | 62-77 | 13 |
| 3 | EPLTHAPRKVYDTRDD | 205-220 | 14 |
| 4 | RTPGLHGDCDDDKYRR | 222-237 | 15 |
| 5 | SDPNLDPLMNPHIRVG | 293-308 | 16 |
| 6 | ALGWLARLLRSRAGSR | 240-255 | 17 |
| 7 | GLGLPLIDEVVSPEPEPLNT | 131-150 | 10 |

The antibody of the present invention may also be referred to as an ‘anti-ZFPL1 antibody,’ ‘humanized anti-ZFPL1 antibody,’ or ‘modified humanized anti-ZFPL1 antibody,’ and is used in the broadest sense in the present invention. Particularly, the antibody includes monoclonal antibodies (including full-length monoclonal antibodies), polyclonal antibodies, multispecific antibodies (e.g., bispecific antibodies), and antibody fragments (e.g., variable regions and other sites of the antibody that exhibit the desired bioactivity (e.g., binding to ZFPL1)).

The antibody of the present invention is an antibody in which a specific amino acid sequence is included in the light-chain and heavy-chain CDRs so that the antibody is capable of selectively binding to ZFPL1 and includes both a monoclonal antibody and a polyclonal antibody, preferably a monoclonal antibody. Moreover, the antibody of the present invention includes all of a chimeric antibody, a humanized antibody, and a human antibody, and is preferably a human antibody.

In the present invention, the term ‘monoclonal’ refers to the properties of an antibody obtained from a population of substantially homogeneous antibodies and does not necessarily mean that the antibody must be produced through any particular method. For example, a monoclonal antibody of the present invention may be produced through the hybridoma method first described in Kohler et al. (1975, Nature 256:495), or through a recombinant DNA method (U.S. Pat. No. 4,816,567). It may also be isolated from phage antibody libraries using, for example, techniques described in the literature (Clackson et al. (1991) Nature 352:624-628 and Marks et al. (1991) J. Mol. Biol. 222:581-597 and Presta (2005) J. Allergy Clin. Immunol. 116:731).

In the preferred embodiment described below, the generated ZFPL1 antibody (also referred to as the “PA1623 antibody”) specifically binds to the 131st to 150th amino acids (SEQ ID NO: 10) of the ZFPL1 protein (SEQ ID NO: 11). The antibody was generated by immunizing mice against synthetic peptide GLGLPLIDEV VSPEPEPLNT (SEQ ID NO 10). The hybridomas were generated, and secreted PA1623 antibodies were tested for binding capacity by ELISA. The PA1623 antibodies were then tested for cross reactivity with prostatic secretions and were found to be specific for ZFPL1. These methods for creating an antibody are well-known in the art and can be easily reproduced by a person of skill in the art to generate the antibodies that bind to the other ZFPL1 epitopes presented in Table 3.

The PA1623 antibody is monoclonal and an immunoglobulin (Ig) G isotype. The antibody comprises a variant Fc domain. Although the entire sequence of PA1623 is disclosed in SEQ ID NO 9, a skilled artisan will recognize that portions of the sequence—outside of the paratope that correlates to the ZFPL1 epitope—may vary and still be effective in the diagnosis and treatment methods disclosed below. In addition, the claimed diagnosis and treatment methods may also be performed by antibodies that bind to any of the other epitopes of ZFPL1 (see Table 3). Each of these antibodies will be monoclonal and immunoglobulin (Ig) G isotypes. Additionally, in order to perform their claimed functions, these antibodies will have an equilibrium dissociation constant (K_{D}) value of approximately 100 nM and a half maximal inhibitory concentration (IC₅₀) of approximately 10 nM.

Development of immunosensor for diagnosis of prostate cancer: An exemplary embodiment of an immunosensor incorporating a ZFPL1 antibody is pictured in FIG. 11 and described below. The immunosensor shown is built on a gold-coated nano AAO chip. The self-assembled monolayers (SAMs) were prepared by performing a series of chemical reactions and then covalently attaching the monoclonal antibodies on the chip surface. First, the chips are incubated with mixed alkanethiol solution containing 5 mM of 11-mercaptoundecanoic acid and 50 mM 8-mercapto-1-octanol. The SAMs were activated by incubating the chip in phosphate buffer containing 2 mM NHS: N-hydroxysuccinimide and 8 mM EDC: N-(3-Dimethylaminopropyl)-N′-ethylcarbodiimide hydrochloride. The chips were then incubated with the novel primary antibodies against ZFPL1 disclosed herein. In the particular embodiment pictured in FIG. 11 and used in the experiments described below, the PA1623 antibody was used. However, it is to be understood that any of the other novel antibodies described in this application could be used as well. Additionally, one skilled in the art will recognize that the particular method for building the inventive ZFPL1 optical immunosensor is merely demonstrative and can easily be modified to adapt to various circumstances.

Although an optical immunosensor was employed in the preferred embodiment disclosed herein, one of skill in the art will recognize that a number of other assay methods may be used to determine ZFPL1 levels in a sample, including chemiluminescent assay, enzyme-linked immunosorbent assay (ELISA), a radioimmunoassay, a dot/blot assay. Additionally, the claimed immunosensor comprises a substrate with which the antibodies of the invention engage. The antibodies can engage with the substrate by, for example, passive adsorption or can be chemically bonded to the substrate attached by way of, for example, covalent bonds. Such covalent bonding generally requires the initial introduction of a chemically active compound covalently attached to the substrate surface prior to antibody addition. The antibody itself may also require the addition of a chemical activating group to achieve substrate bonding. These requirements are well known in the art. The substrate can be any medium capable of adsorbing or bonding to an antibody, for example a bead or nanoparticle (optionally chemically activated) but is preferably of a planar conformation (optionally chemically activated) such as a microtitre plate or biochip. A biochip is a thin, wafer-like substrate with a planar surface which can be made of any suitable material such as glass or plastic but is preferably made of ceramic. The biochip is able to be chemically activated prior to antibody bonding or is amenable to the passive adsorption of antibodies.

Turning back to the preferred immunosensor shown in FIG. 11, the immunosensors were washed, blocked, and incubated with known concentrations of ZFPL1 peptides or unknown serum samples for approximately one hour, and not less than 30 minutes. The chips were then thoroughly washed and dried. The readouts were taken on an optical detector as follows: after the biochip had been functionalized and the antibody had bound to the surface, a white-light source was collimated by a lens to illuminate the chip. The transducing signals are the reflected optical interference fringes, which are detected by an optical detector. This reading served as a blank. After the sample(s) had been applied to the sensor, the transducing signals shifted, due to the binding of the antibody and biomarker, and reflected a different signal. Data from peaks ranging from 550 nm to 750 nm were used to measure the average shifts. FIG. 12 demonstrates the wavelength shifts recorded during testing of the inventive immunosensor disclosed herein. In FIG. 12A, the solid line represents the reading of the blank with only the antibodies, and the dotted line represents the reading of the chip with ZFPL1 applied. In FIG. 12B, the solid line represents the reading of the blank with only the antibodies, and the dotted line represents the reading of the chip with bovine serum albumin (BSA) applied as a control. As is visible from the graphs in FIG. 12, no optical shift is observed when BSA is used, but there is significant wavelength shift produced by ZFPL1.

Turning now to FIG. 13, the immunosensor was tested for ultra-sensitivity and high specificity for the detection of ZFPL1 and PSA in sera of patients. The immunosensor-based assay is label-free. Unlabeled synthetic peptides of partial sequence were used as a reference for both antigens. The assay was tested for accuracy, precision, recovery, and linearity. The dilution curve of human serum was parallel to the ZFPL1 standard curve in the range of 0.1-2 μl serum. The test included pools of negative (serum pool from patients who have undergone prostatectomy; serum PSA <0.003 ng/ml) and positive samples (serum pool of prostate cancer patients confirmed by biopsy). At present, intra- and inter-assay variations of the assay are less than 5% and 9% respectively. To compare the efficacy of the immunosensor with ELISA, we compared the ZFPL1 Calibration curves by both systems. The immunosensor (FIG. 13B) was approximately 50-fold more sensitive than the corresponding nonequilibrium ELISA (FIG. 13A). The assay is linear over the range of 1-64 pg with a sensitivity of 1 pg/50 μl. Thus, serum ZFPL1 levels can be measured in as little as 1 pg of ZFPL1/mL of serum. Thus, when considered together with the simplicity of the method—a label-free assay with a short incubation period of 90 minutes—the presently disclosed immunosensor offers significant advantages over the ELISAs for ZFPL1 measurements.

Presence of ZFPL1 in sera of patients with prostate cancer: Since ZFPL1 was secreted by prostate cancer cells, its presence in sera of healthy volunteers and patients with prostate cancer was examined next. PSA was used as a reference biomarker in same cohort. The serum profiles of ZFPL1 and PSA in healthy donors and positively confirmed patients with prostate cancer are presented in FIG. 10. The scattergram demonstrates that serum ZFPL1 levels in non-cancer individuals (controls; mean±SEM 3.6±0.286 ng/ml, n=36) were significantly lower than all patients with prostate cancer (cancer: 11.41±0.6135, n=75) with no overlap (P<0.0001, unpaired t-test). By contrast, PSA levels displayed significant overlap between non-cancer and cancer patients (controls: 6.26±0.9, n=37 vs. Cancer: 22.85±2.96, n=42, not significant by unpaired t-test). This distinct separation of ZFPL1 levels in non-cancer and cancer patients suggested that the ZFPL1 test will significantly improve the specificity of prostate cancer detection. Additionally, this data suggests that a ZFPL1 concentration of approximately 3.3 ng/ml or more correlates with a positive prostate cancer diagnosis.

Predictability of ZFPL1 immunosensor: Next, serum ZFPL1 and PSA levels in healthy donors (n=119) and prostate cancer patients (n=205) were measured by the inventive immunosensor assay, and the diagnostic potential of both markers was quantified using the area under the receiver operating characteristic curve (AUC). These are also reflected in prediction curves for normal (0) vs prostate cancer (1). As is shown in FIG. 14A, the AUC of ZFPL1 was 0.9788 (extremely close to perfect 1.0) with a P<0.0001. Further, the mean ZFPL1 value of normal individuals was clearly different from that of prostate cancer patients. In contrast, viewing FIG. 14B, the AUC value for PSA was 0.9055, and there was significant overlap between the normal and prostate cancer samples. Finally, as is shown in FIG. 14C, when the data from the two markers was combined, the AUC was 0.9793.

Tables 4 and 5 summarize the accuracy of the immunoassay and method of diagnosis when testing for ZFPL1 and PSA. The results show that the ZFPL1 immunoassay correctly predicted whether serum was positive for cancer 92.59%, compared to only 84.88% accuracy for the PSA immunoassay, suggesting the ZFPL1 immunoassay remarkably decreases inaccurate diagnoses. Negative predictability is important to study in addition to positive predictability as it can prevent unnecessary follow-up testing, such as biopsies.

TABLE 4

| | Negative | Positive | | % Correctly |
|---|---|---|---|---|
| | Result | Result | Total | Predicted |
| Healthy Donors | 112 | 7 | 119 | 94.12 |
| Prostate Cancer | 17 | 188 | 205 | 91.71 |
| Donors | | | | |
| Total | 129 | 195 | 324 | 92.59 |

TABLE 5

| | Negative | Positive | | % Correctly |
|---|---|---|---|---|
| | Result | Result | Total | Predicted |
| Healthy Donors | 95 | 24 | 119 | 79.83 |
| Prostate Cancer | 25 | 180 | 205 | 87.80 |
| Donors | | | | |
| Total | 120 | 204 | 324 | 84.88 |

Table 6 provides the mean±SD of ZFPL1 and PSA levels in stratified conditions from the same cohort, as determined by the ZFPL1 immunosensor disclosed herein. ZFPL1 clearly discriminates prostate cancer from other prostate diseases and shows very high levels in cases of metastatic disease. This test can be useful in monitoring patients after therapy to check for tumor relapse (biochemical recurrence or BCR).

TABLE 6

| Condition | n | PSA (ng/mL) | ZFPL1 (ng/mL) |
|---|---|---|---|
| Normal | 61 | 1.795 ± 2.141 | 1.204 ± 2.143 |
| BPH | 10 | 5.396 ± 3.629 | 3.096 ± 2.244 |
| Prostate Cancer | 79 | 15.30 ± 38.18 | 59.09 ± 6.912 |
| BCR | 11 | 3.885 ± 7.253 | 5.615 ± 6.912 |
| Mets/Death | 3 | 1871 ± 721.7 | 1661 ± 742.4 |
| Other Cancers | 44 | 8.295 ± 4.858 | 10.72 ± 5.712 |

Predictability in the gray zone: One of the weaknesses of diagnosis using PSA is that PSA has a high diagnostic sensitivity but a relatively low specificity, which can lead to overdiagnosis and treatment of indolent prostate lesions. In particular, only 22% of patients had a positive prostate biopsy when the PSA values ranged between 4 ng/ml and 10 ng/ml, also known as the “gray zone.” To investigate the effectiveness of the ZFPL1 test in the gray zone, ROC curves of ZFPL1 and PSA levels in patients with serum levels in the gray zone were prepared. The results show that the predictability of PSA (FIG. 15B) in this sample population is very low, with an AUC value of 0.770, whereas the predictability of ZFPL1 (FIG. 15A) in the same population has an AUC value of 0.9672. The ZFPL1 immunosensor correctly predicted that 19 out of 23 patients did not have cancer, and they need not undergo biopsy/therapy, with an NPV of 82.6. The ZFPL1 test's PPV was 95.5, correctly predicting that 87 out of 88 patients did have cancer.

Effect of ZFPL1 monoclonal antibody on prostate cancer cell growth: In order to examine the potential of ZFPL1 antibodies as a druggable target, two prostate cancer cell lines, PC3-CTR and DU-145, were cultured and incubated with 0 μl, 0.25 μl, 0.5 μl, or 1 μl of PA1623 ZFPL1 antibody. PC3-CTR and DU-145 were selected because of their ability to grow rapidly in androgen-independent conditions. The cell growth was measured as optical density at 600 nm (OD₆₀₀), and the results are summarized in FIG. 16. Both cell lines showed reduced cell growth at all treatment amounts compared to the cells that received no ZFPL1 antibody treatment. The results illustrate the potent, dose-related inhibiting effect of the ZFPL1 antibody on the growth of PC3-CTR as well as DU-145 cells. The antibody's inhibition of the growth of these highly invasive cell lines presents promising potential for the use of the antibody as an effective therapeutic agent for castration-resistant prostate cancer.

Effect of ZFPL1 monoclonal antibody as a treatment in animal model: Next, follow up experimentation was conducted to test the effectiveness of ZFPL1 antibodies as a treatment in an animal model. In the study, adult nude mice weighing approximately 30 g were injected with a PC3-CTR cell suspension—Matrigel™ mix (1:1 v/v)—in their flanks. The mice were then observed daily. The tumors became observable 11-14 days after cell implantation. Their observation, treatment with PA1623 ZFPL1 antibody, and tumor volume measurement by calipers were started approximately two weeks after the implantation. The infected mice either received no treatment (“untreated” group), saline solution (“control” group), 1 μl of antibody solution (containing 4.5 μg of ZFPL1 antibody), or 2 μl of antibody solution (containing 9 μg of ZFPL1 antibody). The ZFPL1 antibody was injected intratumorally every Monday and Thursday until termination of the study. The “Untreated” group was left untouched after PC3-CTR cell implantation, whereas the “Control” group was injected with saline on the same schedule as the ZFPL1 antibody injections in the other groups.

FIG. 17 is a scatterplot of the tumor volume from day 15 after the cell implantation. The results show that treatment with 4.5 μg ZFPL1 Antibody remarkably slowed down tumor growth, and the tumor-growth suppression was even greater at the higher dose of 9 μg. FIG. 18 includes representative images of tumors (untreated and treated) and a graph of their weights at the time of necropsy. A significant difference in the appearance and texture of the tumors was observed. For example, the tumor of the untreated mouse was extremely hard, as expected. In contrast, the tumors of the treated mice were very soft and had a strong smell of narcotic tissue. These results demonstrate the effectiveness of the ZFPL1 antibody in slowing tumor growth and therefore treating prostate cancer.

Based on the totality of the results presented above, it is expected that a preferred embodiment of human treatment will involve administration of 0.5-5.0 mg of antibody/kg of body weight. The treatment is preferably administered intraperitoneally, but in other embodiments may be administered intratumorally, intravenously, or subcutaneously. Because the half-life of antibodies in humans is typically 3-4 weeks, the treatment will preferably be administered approximately every 2-3 days for a week, followed by no treatment for approximately three weeks. This process can be repeated until the patient's ZFPL1 and/or PSA levels have reached a normal state or until the patient's tumor has decreased in size to a point where it can be removed surgically.

The administration of the monoclonal antibody as described herein may lead to inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, and/or stable disease. This treatment method can be combined with one or more of surgery, radiation, a chemotherapeutic agent, a cancer vaccine, an antibody-drug conjugate, an anti-inflammatory drug, a dietary supplement, or any other treatment for prostate cancer and/or its symptoms that is currently known or may be developed in the future.

The foregoing description and accompanying drawings illustrate the principles, exemplary embodiments, and modes of operation of the invention. However, the invention should not be construed as being limited to the particular embodiments discussed above. Many modifications of the embodiments described herein will come to mind to one skilled in the art having the benefit of the teaching presented in the foregoing descriptions and the associated drawings. Accordingly, it should be appreciated that variations to those embodiments can be made by those skilled in the art without departing from the scope of the invention.

What is claimed is:

1. A method of diagnosing and treating prostate cancer in a mammalian patient, the method comprising:
a. obtaining a serum sample from the patient;
b. detecting whether zinc finger protein-like 1 (ZFPL1) is present in the serum sample;
c. diagnosing the patient with prostate cancer when the presence of ZFPL1 in the serum sample is detected; and
d. administering an effective amount of anti-ZFPL1 antibodies to the diagnosed patient.

2. The method of claim 1, wherein the level of the ZFPL1 is determined using a label-free optical immunoassay comprising a monoclonal antibody, capable of specifically binding to ZFPL1, wherein the monoclonal antibody is immobilized on a substrate.

3. The method of claim 1, wherein the monoclonal antibody specifically binds to a protein that comprises an epitope selected from a group consisting of SEQ ID No: 10, SEQ ID No: 12, SEQ ID No: 13, SEQ ID No: 14, SEQ ID No: 15, SEQ ID No: 16, or SEQ ID No: 17.

4. The method of claim 1, wherein the monoclonal antibody comprises:
a. a heavy chain variable region (HCVR) comprising heavy chain complementarity-determining regions (CDRs), the heavy chain CDRs comprising SEQ ID Nos: 1, 2, and 3; and
b. a light chain variable region (LCVR) comprising light chain CDRs, the light chain CDRs comprising SEQ ID Nos: 4, 5, and 6.

5. The method of claim 1, wherein the monoclonal antibody comprises the HCVR (SEQ ID NO: 7) and the LCVR (SEQ ID NO:8).

6. The method of claim 1, wherein the isotype of the monoclonal antibody is immunoglobulin G.

7. The method of claim 1, wherein the antibody has an equilibrium dissociation constant (K_{D}) value of 100 nM and a half maximal inhibitory concentration (IC₅₀) of 10 nM.

8. The method of claim 1, wherein the patient is diagnosed with prostate cancer when the serum sample has a level of ZFPL1 greater than or equal to 3.3 ng/mL.

9. The method of claim 1, wherein the monoclonal antibody treatment is administered intravenously, subcutaneously, or intraperitoneally.

10. The method of claim 9, wherein the monoclonal antibody treatment is administered intraperitoneally.

11. The method of claim 1, wherein the monoclonal antibody treatment leads to at least one effect selected from the group consisting of inhibition of tumor growth, tumor regression, reduction in the size of a tumor, reduction in tumor cell number, delay in tumor growth, abscopal effect, inhibition of tumor metastasis, reduction in metastatic lesions over time, reduced use of chemotherapeutic or cytotoxic agents, reduction in tumor burden, increase in progression-free survival, increase in overall survival, complete response, partial response, and stable disease.

12. The method of claim 1, further comprising administering to the patient an additional therapeutic agent or therapy, wherein the additional therapeutic agent or therapy is selected from the group consisting of surgery, radiation, a chemotherapeutic agent, a cancer vaccine, an antibody-drug conjugate, an anti-inflammatory drug, and a dietary supplement.

13. A method of treating prostate cancer in a patient, the method comprising administering an effective amount of anti-ZFPL1 antibodies to a patient suffering from prostate cancer.

14. The method of claim 13, wherein the anti-ZFPL1 antibody is a monoclonal antibody, an antigen-binding fragment thereof, or a protein ligand.

15. The method of claim 13, wherein the anti-ZFPL1 antibody binds to at least four nucleotides within the nucleotide positions of:
a. 269-284;
b. 62-77;
c. 205-220;
d. 222-237;
e. 293-308:
f. 240-255; or
g. 131-150;
within SEQ ID NO: 11.

16. The method of claim 13, wherein the anti-ZFPL1 antibody binds to at least four nucleotides within the nucleotide positions of 62-77, 127-284 or 293-308 within SEQ ID NO: 11.

17. The method of claim 13, wherein the anti-ZFPL1 antibody binds to at least seven nucleotides within the nucleotide positions of 62-77, 127-284 or 293-308 within SEQ ID NO: 11.

18. The method of claim 13, wherein the anti-ZFPL1 antibody is a chimeric or humanized antibody.

19. The method of claim 13, wherein the anti-ZFPL1 antibody comprises a variant Fc domain.

20. The method of claim 13, wherein the anti-ZFPL1 antibody is an isolated monoclonal antibody or antigen-binding portion thereof comprising:
a. an HCDR1 comprising an amino acid sequence with amino acid substitutions at the 2nd, 4th, or both the 2nd and 4th amino acids of SEQ ID NO:1;
b. an HCDR2 comprising an amino acid sequence of SEQ ID NO:2;
c. an HCDR3 comprising an amino acid sequence with amino acid substitutions at the 8th amino acids of SEQ ID NO:3;
d. an LCDR1 comprising an amino acid sequence with amino acid substitutions at the 8th, 11th, 13th, or all of these amino acids of SEQ ID NO:4,
e. an LCDR2 comprising an amino acid sequence with amino acid substitutions at the 4th of SEQ ID NO:5; and
f. an LCDR3 comprising an amino acid sequence with amino acid substitutions at the 4th 7th, or both the 4th and 7th amino acids of SEQ ID NO:6;
g. and wherein said antibody or antigen-binding portion thereof binds to the polypeptide of SEQ ID NO:11 with a K_{D} of 100 nM or less.
